(19) United States
(12) Patent Application Publication
(10) Pub. No.: US 2025/0258190 A1
(43) Pub. Date: Aug. 14, 2025

(54) MULTIPLE LANE EXTRACTION DRAWER AND PLATE SEALER

(71) Applicant: BECTON, DICKINSON AND COMPANY, Franklin Lakes, NJ (US)

(72) Inventors: Stephen Robert LaChance, Cockeysville, MD (US); Alyssa Nicolaisen Shedlosky, Reiserstown, MD (US); Jacob Monroe, Centerville, MA (US); David Cleary, Centerville, MA (US); Steven Conrad, Centerville, MA (US)

(21) Appl. No.: 19/196,151

(22) Filed: May 1, 2025

Related U.S. Application Data
(63) Continuation of application No. PCT/US2023/078313, filed on Nov. 1, 2023.
(60) Provisional application No. 63/422,742, filed on Nov. 4, 2022.
(60) Provisional application No. 63/585,895, filed on Sep. 27, 2023.

(51) Int. Cl. G01N 35/04; G01N 35/00

(52) U.S. Cl. CPC G01N 35/04; G01N 35/0099; G01N 2035/0403

(57) ABSTRACT

An automated analyzer can include a plate sealer and a drawer assembly. The plate sealer can include a heated press plate configured to apply heat and pressure to a seal material, wherein the seal material is configured to seal against a top surface of an amplification plate. The plate sealer can include a knife assembly configured to cut the seal material, wherein the heated press plate and the knife assembly are configured to be lowered simultaneously to cut and seal the seal material. The drawer assembly can include a two lane drawer comprising a pipette tip station configured to house two pipette tip holders, an extraction container station configured to house two extraction container holders, and an amplification plate station configured to house two amplification plates. The drawer assembly can include an extractor module positioned relative to the extraction container station, wherein the extractor module comprises magnets configured to apply a magnetic field to extraction container holders of the two extraction container holders.

### CROSS REFERENCE TO RELATED APPLICATIONS

This application is a continuation of International Application No. PCT/US2023/078313, filed Nov. 1, 2023, which claims the benefit of U.S. Provisional Application No. 63/422,742, filed Nov. 4, 2022, and U.S. Provisional Application No. 63/585,895, filed Sep. 27, 2023, all of which are hereby incorporated by reference in their entirety.

### BACKGROUND

### Field

The technology described herein generally relates to an automated diagnostic analyzer including components thereof. In some aspects, the technology more particularly relates to a multiple lane extraction drawer. In some aspects, the technology relates to a plate sealer.

### Description of the Related Art

Diagnostic testing of biological samples is instrumental in the health care industry's efforts to quickly and effectively diagnose and treat disease. Clinical laboratories that perform such diagnostic testing already receive hundreds or thousands of samples on a daily basis with an ever increasing demand. The challenge of managing such large quantities of samples has been assisted by the automation of sample analysis. Automated sample analysis is typically performed by automated analyzers that are commonly self-contained systems which perform multistep processes on the biological samples to obtain diagnostic results.

Several current automated clinical analyzers offer a user an array of automated tests or assays that can be performed on a provided sample. Additionally, when samples arrive at the laboratory, they are often not ready for analysis. In order to prepare a sample for testing with an automated analyzer, a lab technician typically transfers an aliquot of the sample from a primary container, as received by the laboratory, to a secondary container which is amenable to the analyzer. In addition, the technician typically must know what tests are to be performed on the sample so that the technician can select a test specific reagent or diluent to be paired with the sample. This can be time consuming and can lead to operator error and exposure to communicable diseases.

Pre-analytical systems meant to help prepare a sample for analysis and further remove the operator from the workflow between the laboratory's receipt of a sample and the analyzer's test results also exist. However, many of these systems still require significant technician involvement, such as: prior to loading samples in the pre-analytical system; after the samples have been prepared by the pre-analytical system; and after the analyzers have completed analysis.

For example, some pre-analytical systems may automatically transfer an aliquot of sample from a first container to a second container. However, such systems often require a technician to manually match identification codes of the first and second containers prior to loading them into the system, which can be time consuming and is prone to error.

In addition, many of these systems are not capable of being integrated with one or more analyzers, and, conversely, the analyzers are not capable of being integrated with such systems. In this regard, a technician must be present to manually transfer the samples from the pre-analytical system to an analyzer and from the analyzer to a storage location once analysis is complete. This requires skilled labor to perform menial tasks and can create distractions in that the technician must be ever mindful of the progress of the samples within the pre-analytical system and analyzer so that the technician is prepared to transfer samples when ready in order to minimize downtime.

Moreover, current pre-analytical systems generally prepare samples at different rates than the analyzers evaluate such samples. This further complicates the integration between pre-analytical systems and analyzers. In this regard, a technician may be required to continuously keep track of samples prepared by the pre-analytical system until a full batch of samples is accumulated for manual transfer to an analyzer. Alternatively, technicians may transfer partial batches to an analyzer, which can reduce the analyzer's productivity.

Thus, while current automated pre-analytical systems and analyzers are beneficial to the clinical laboratory, there is room for better integration and automation of various systems.

### SUMMARY

The present disclosure describes devices, systems, and methods for sample processing and analysis.

In some embodiments, a plate sealer is provided. The plate sealer can include a heated press plate configured to apply heat and pressure to a seal material. In some embodiments, the seal material is configured to seal against a top surface of an amplification plate. The plate sealer can include a knife assembly configured to cut the seal material. In some embodiments, the heated press plate and the knife assembly are configured to be lowered simultaneously to cut and seal the seal material.

In some embodiments, the plate sealer can include a roll of the seal material. In some embodiments, the plate sealer can include a rear clamp assembly and a front clamp assembly, wherein the rear clamp assembly and the front clamp assembly are configured to clamp the material when the heated press plate and the knife assembly are lowered. In some embodiments, the plate sealer can include a feed plate configured to advance the seal material. In some embodiments, the plate sealer can include a motor assembly configured to raise and lower the heated press plate and the knife assembly. In some embodiments, the plate sealer can include the amplification plate. In some embodiments, the plate sealer can include a multifunctional robot configured to position the amplification plate. In some embodiments, the plate sealer can include a movable platform configured to receive the amplification plate. In some embodiments, the plate sealer can include a hinged door.

In some embodiments, a two lane drawer assembly is provided. The two lane drawer assembly can include a two lane drawer. The two lane drawer can include a pipette tip station configured to house two pipette tip holders. The two lane drawer can include an extraction container station configured to house two extraction container holders. The two lane drawer can include an amplification plate station configured to house two amplification plates. The two lane drawer assembly can include an extractor module positioned relative to the extraction container station. In some embodiments, the extractor module comprises magnets configured to apply a magnetic field to extraction container holders of the two extraction container holders.

In some embodiments, the extractor module is configured to process samples from the two extraction container holders simultaneously. In some embodiments, the extractor module is configured to move up and down relative to the two extraction container holders. In some embodiments, the extractor module is configured to move up and down along one or more rails. In some embodiments, the two lane drawer comprises a retention feature configured to extend over a portion of the pipette tip station and the extraction container station. In some embodiments, the two lane drawer comprises a latching mechanism comprising a push-to-close latch. In some embodiments, the two lane drawer comprises a kick-out mechanism configured to facilitate the sliding extension of the two lane drawer. In some embodiments, the two lane drawer comprises telescoping slides. In some embodiments, the two lane drawer assembly comprises the two pipette tip holders, the two extraction container holders, and the two amplification plates. In some embodiments, the two lane drawer assembly comprises three of the two lane drawers. In some embodiments, the two lane drawer assembly comprises a tip drawer configured to house five pipette tip holders.

In some embodiments, a plate sealer for use in a diagnostic testing apparatus is provided. The plate sealer can include a heated press plate configured to apply heat and pressure to a seal material. In some embodiments, the seal material is configured to seal against a top surface of an amplification plate. In some embodiments, the amplification plate comprises a plurality of wells that define amplification compartments for amplification. The plate sealer can include a knife assembly configured to cut the seal material. In some embodiments, the heated press plate and the knife assembly are configured to be lowered simultaneously relative to the amplification plate to cut the seal material and seal the seal material to the amplification plate.

In some embodiments, the plate sealer can include a roll of the seal material. In some embodiments, the plate sealer can include a rear clamp assembly and a front clamp assembly, wherein the rear clamp assembly and the front clamp assembly are configured to clamp the seal material when the heated press plate and the knife assembly are lowered. In some embodiments, the plate sealer can include a feed plate configured to advance the seal material. In some embodiments, the plate sealer can include a motor assembly configured to raise and lower the heated press plate and the knife assembly. In some embodiments, the plate sealer can include the amplification plate. In some embodiments, the plate sealer can include a multifunctional robot configured to position the amplification plate. In some embodiments, the plate sealer can include a movable platform configured to receive the amplification plate. In some embodiments, the plate sealer can include a hinged door.

In some embodiments, a two lane drawer assembly is provided. The two lane drawer assembly can include a two lane drawer. The two lane drawer can include a pipette tip station configured to house two pipette tip holders. The two lane drawer can include an extraction container station configured to house two extraction container holders. The two lane drawer can include an amplification plate station configured to house two amplification plates. The two lane drawer assembly can include an extractor module positioned relative to the extraction container station. In some embodiments, the extractor module comprises magnets configured to apply a magnetic field to extraction container holders of the two extraction container holders.

In some embodiments, the extractor module is configured to process samples from the one extraction container holder. In some embodiments, the extractor module is configured to move up and down relative to the two extraction container holders. In some embodiments, the extractor module is configured to move up and down along one or more rails. In some embodiments, the two lane drawer comprises a retention feature configured to extend over a portion of the pipette tip station and the extraction container station. In some embodiments, the two lane drawer comprises a latching mechanism comprising a push-to-close latch. In some embodiments, the two lane drawer comprises a kick-out mechanism configured to facilitate the sliding extension of the two lane drawer. In some embodiments, the two lane drawer comprises telescoping slides. In some embodiments, the two lane drawer assembly can include the two pipette tip holders, the two extraction container holders, and the two amplification plates. In some embodiments, the two lane drawer assembly can include three of the two lane drawers. In some embodiments, the two lane drawer assembly can include a tip drawer configured to house five pipette tip holders.

### BRIEF DESCRIPTION

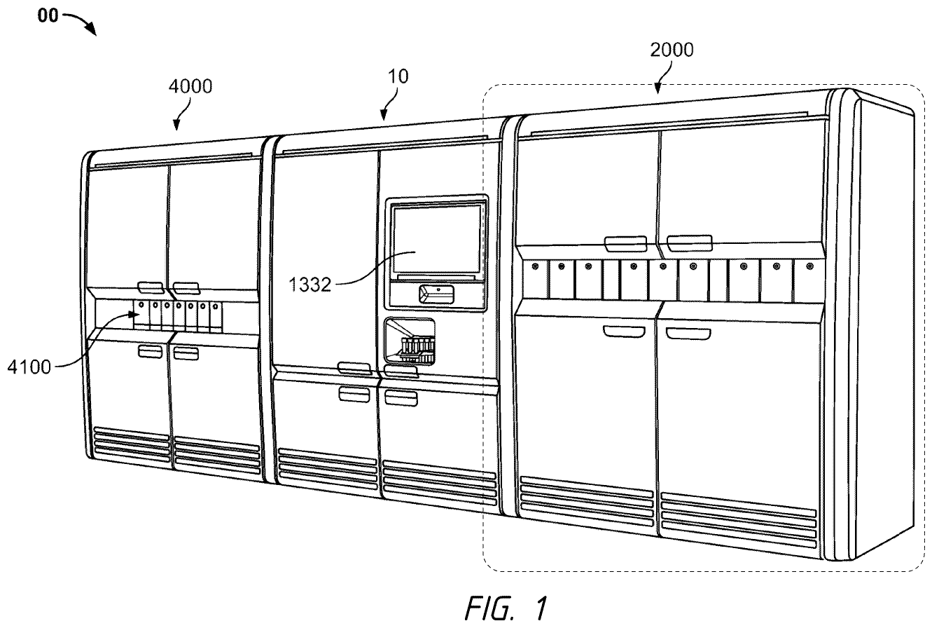

FIG. 1 is a front perspective view of a high-throughput diagnostic system according to an embodiment of the present disclosure.

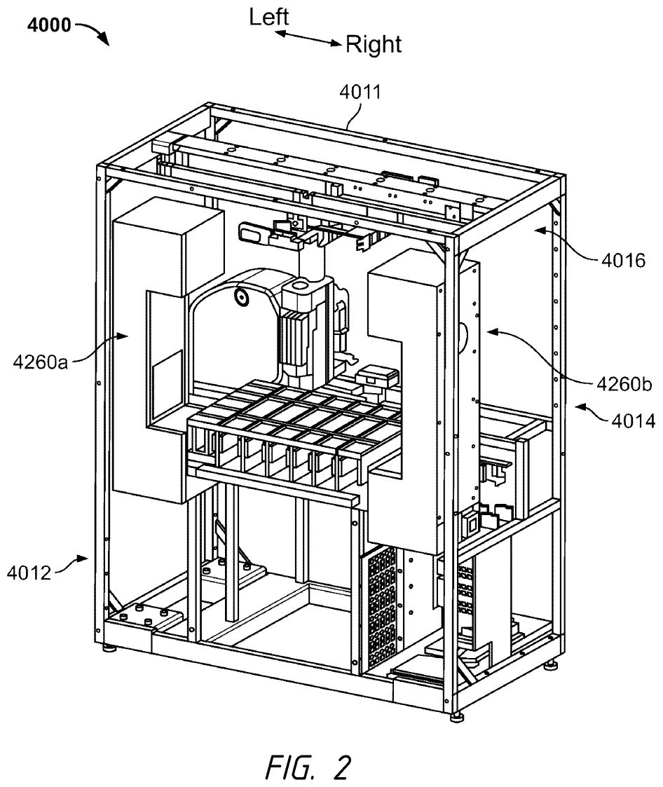

FIG. 2 is a front perspective view of an analyzer of the high-throughput diagnostic system of FIG. 1 absent an external housing.

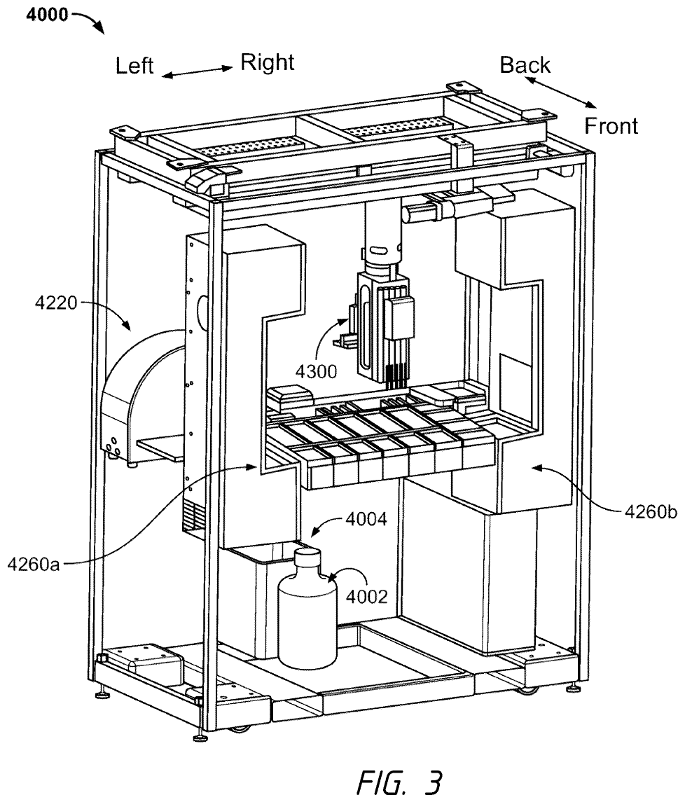

FIG. 3 is another front perspective view of the analyzer of FIG. 2.

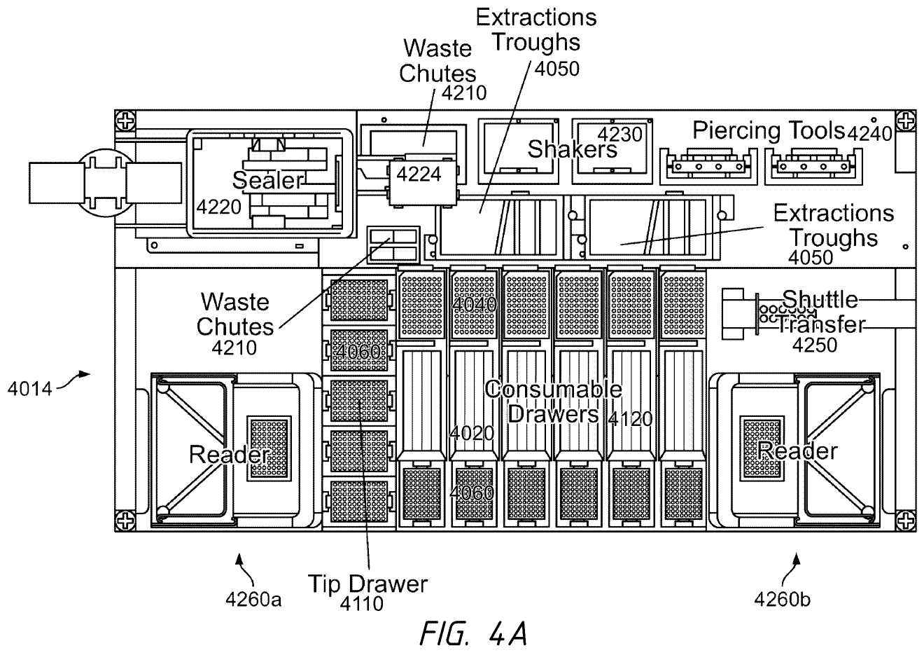

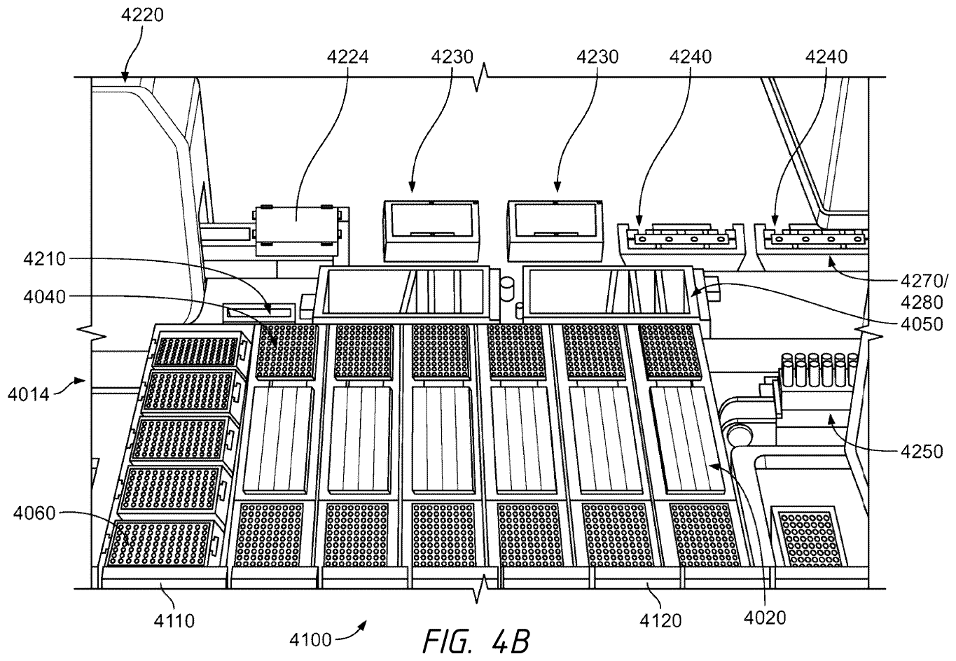

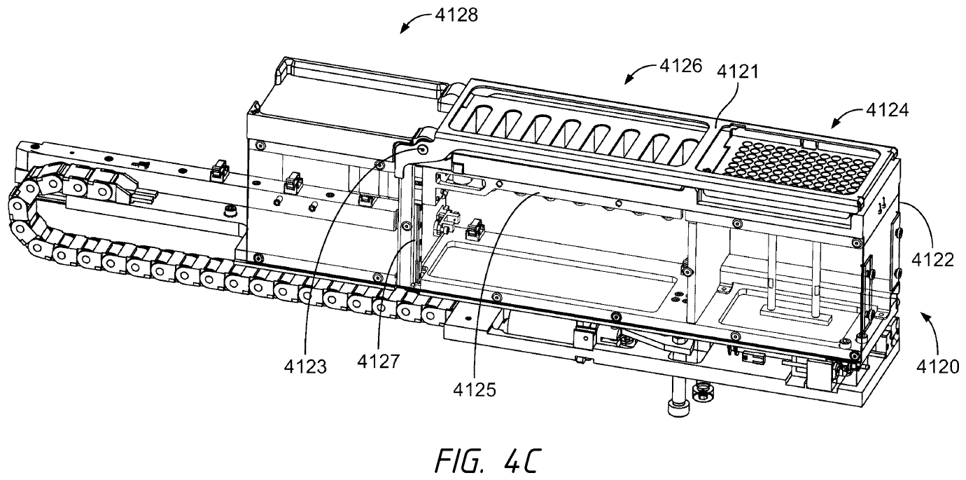

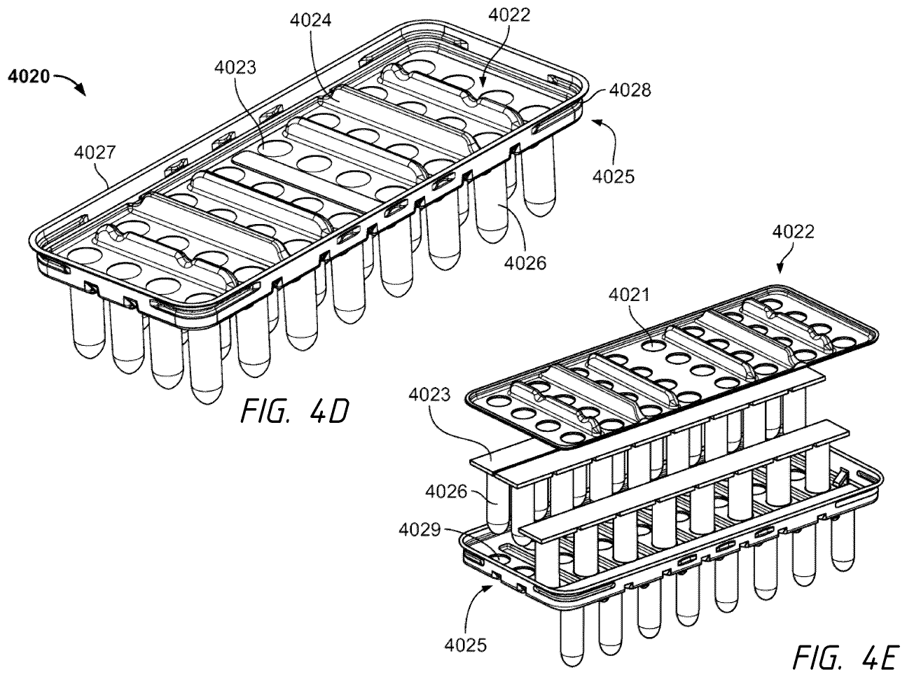

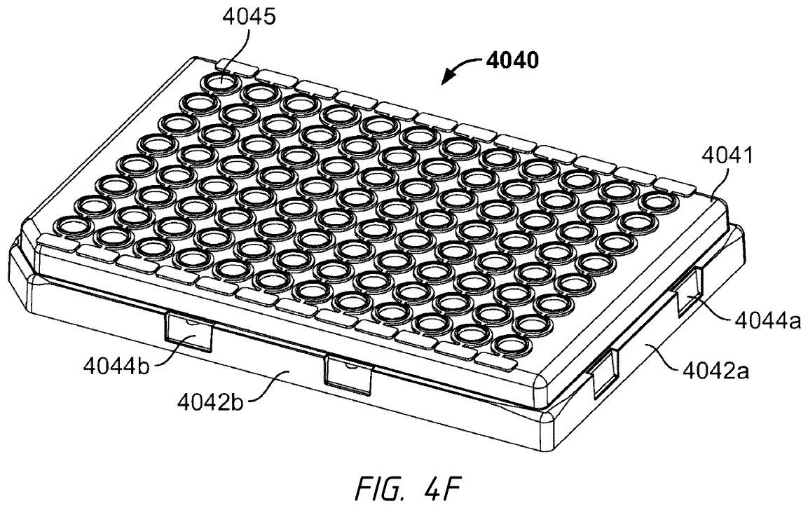

FIG. 4A-4F are views of a processing deck of the analyzer of the high-throughput diagnostic system of FIG. 1, and components thereof.

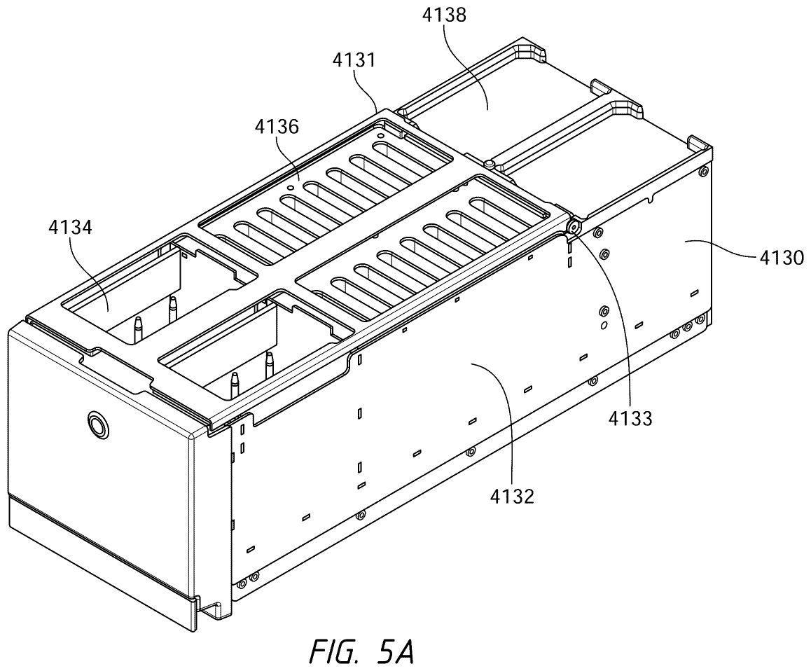

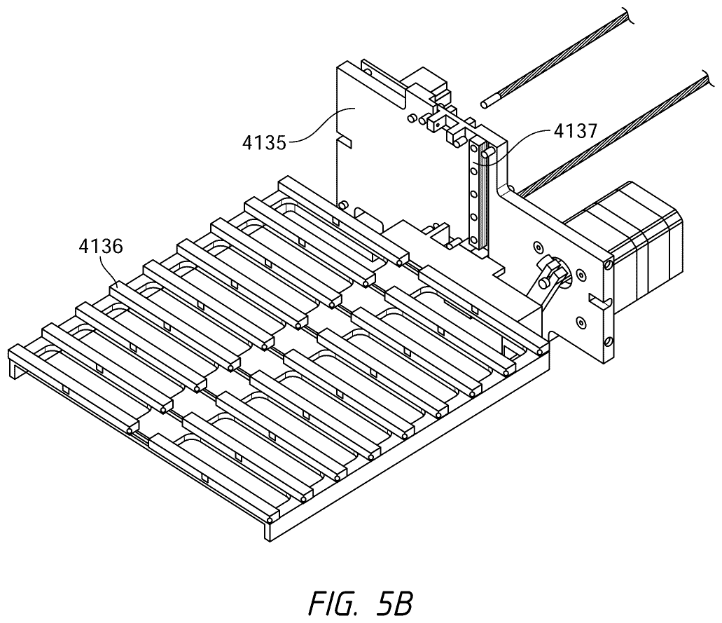

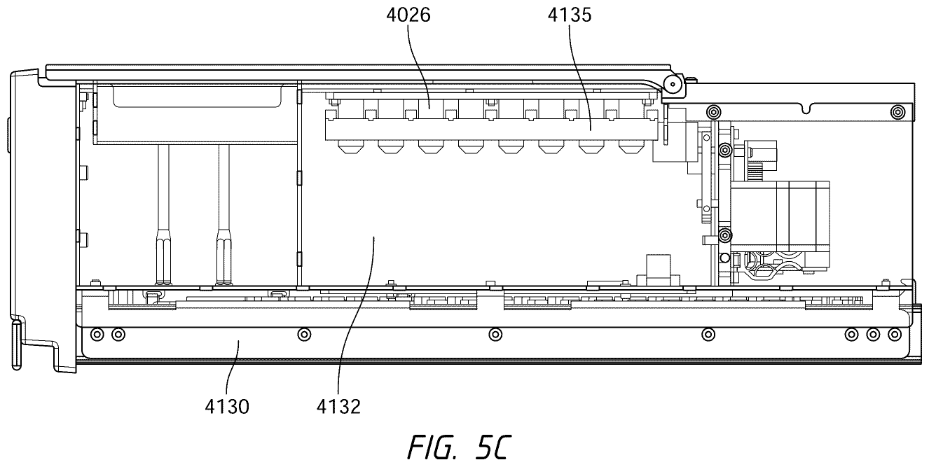

FIGS. 5A-5C are views of an embodiment of a two lane drawer and extractor according to the present disclosure.

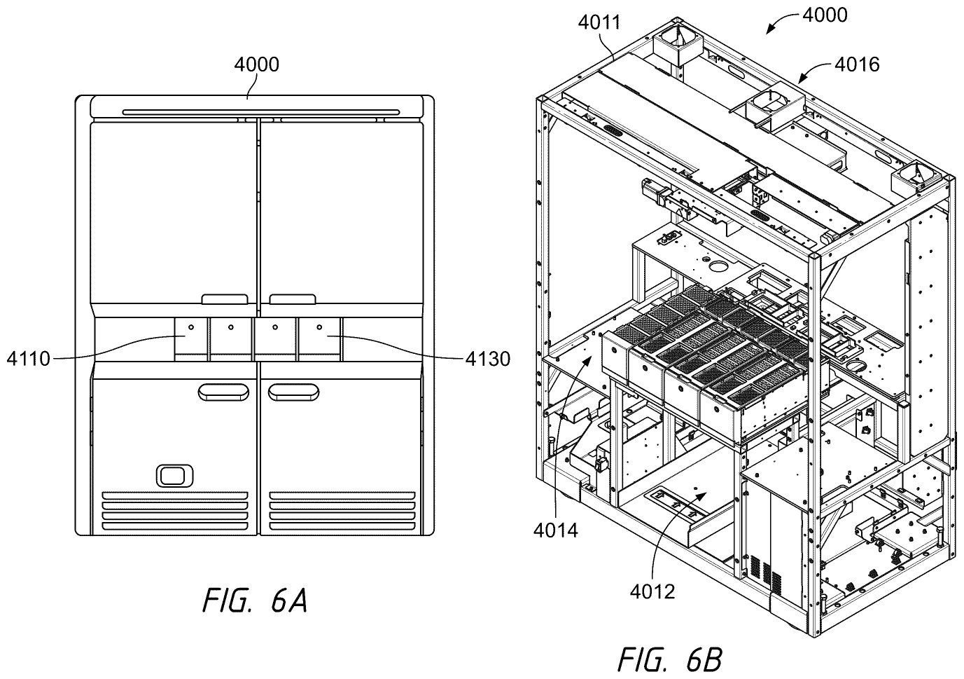

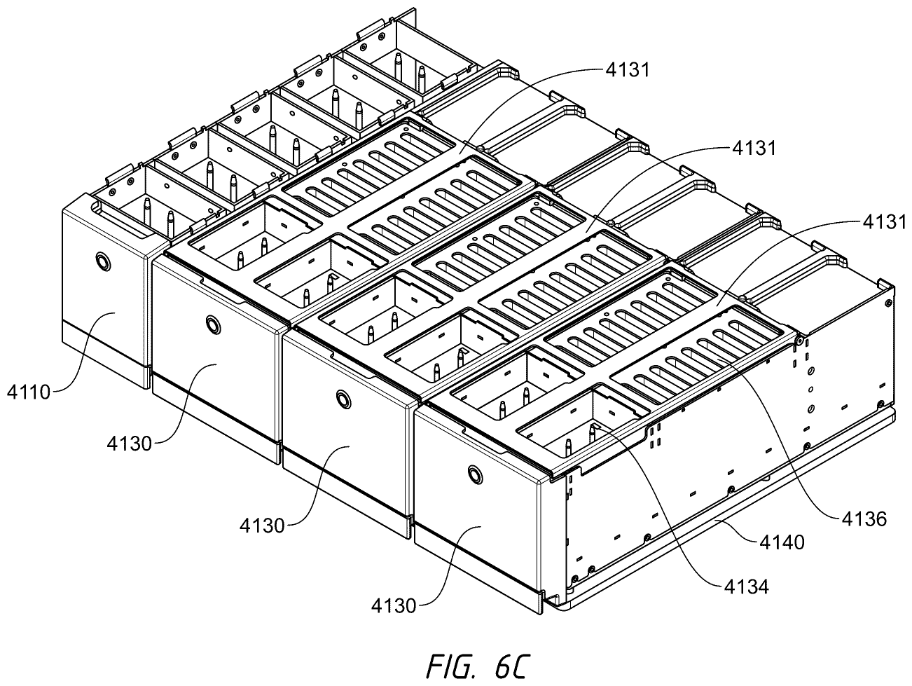

FIGS. 6A-6C are view of the two lane drawer of FIG. 5A in relation to the analyzer according to the present disclosure.

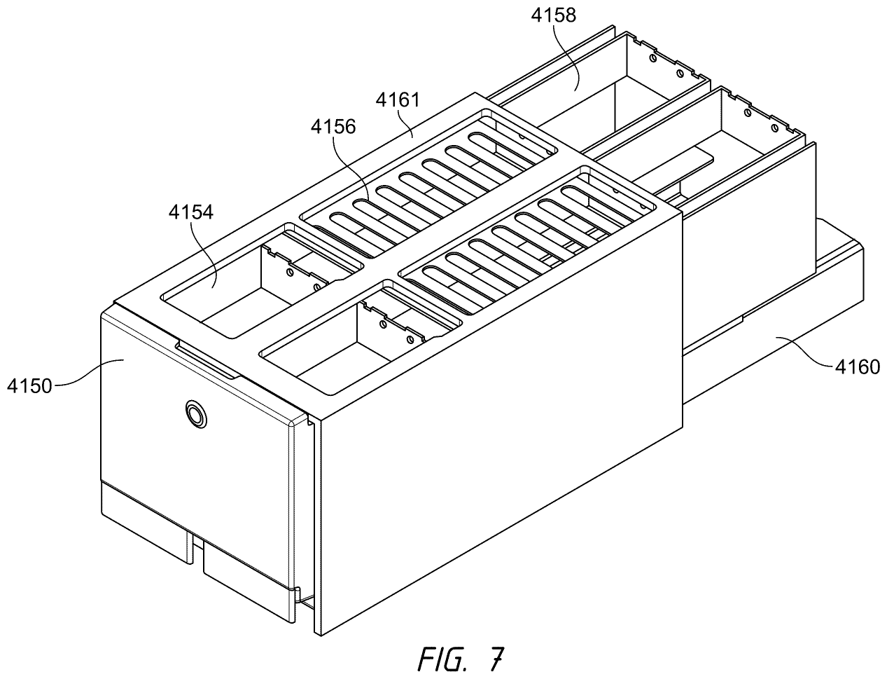

FIG. 7 is a view of an embodiment of a two lane drawer according to the present disclosure.

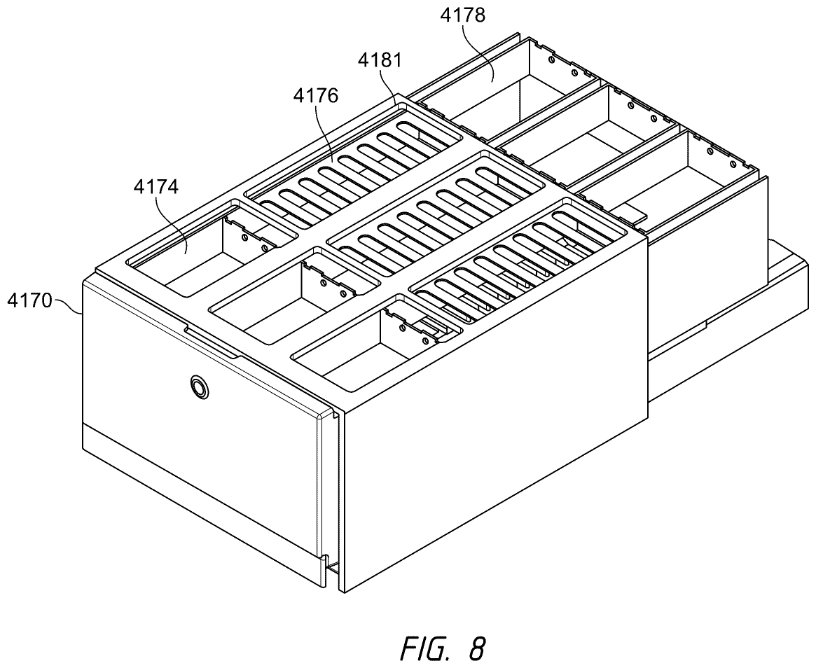

FIG. 8 is a view of an embodiment of a three lane drawer according to the present disclosure.

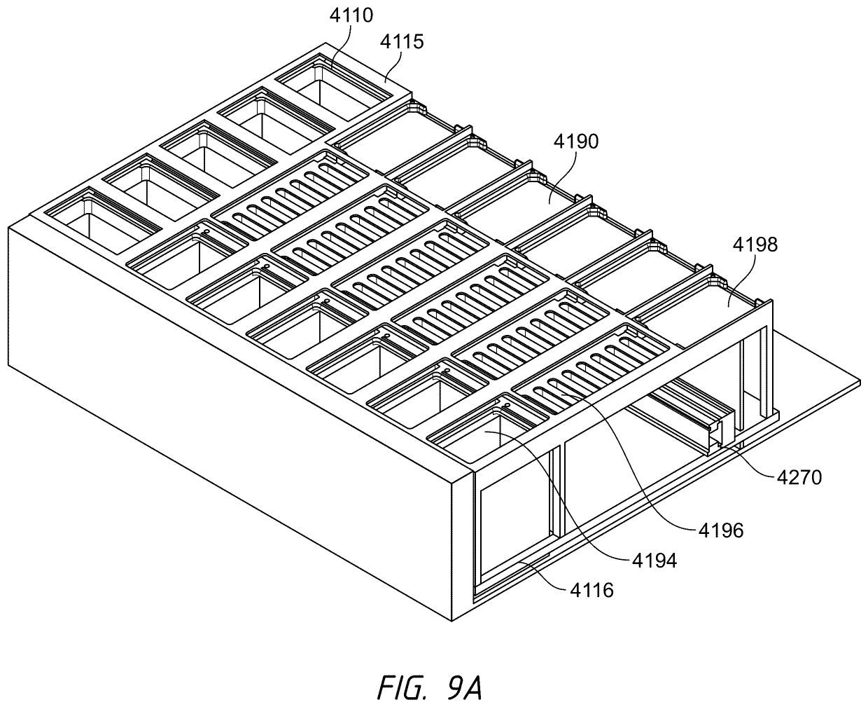

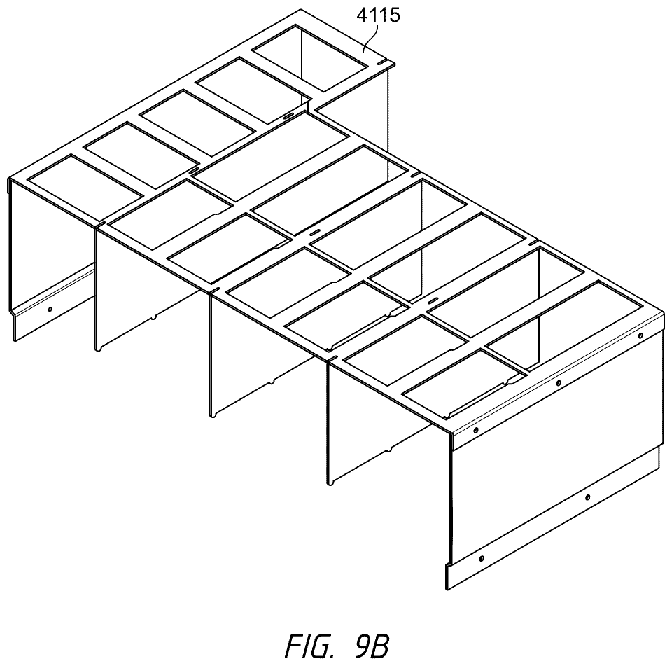

FIGS. 9A-9B are views of an embodiment of a six lane drawer and a stationary retention housing according to the present disclosure.

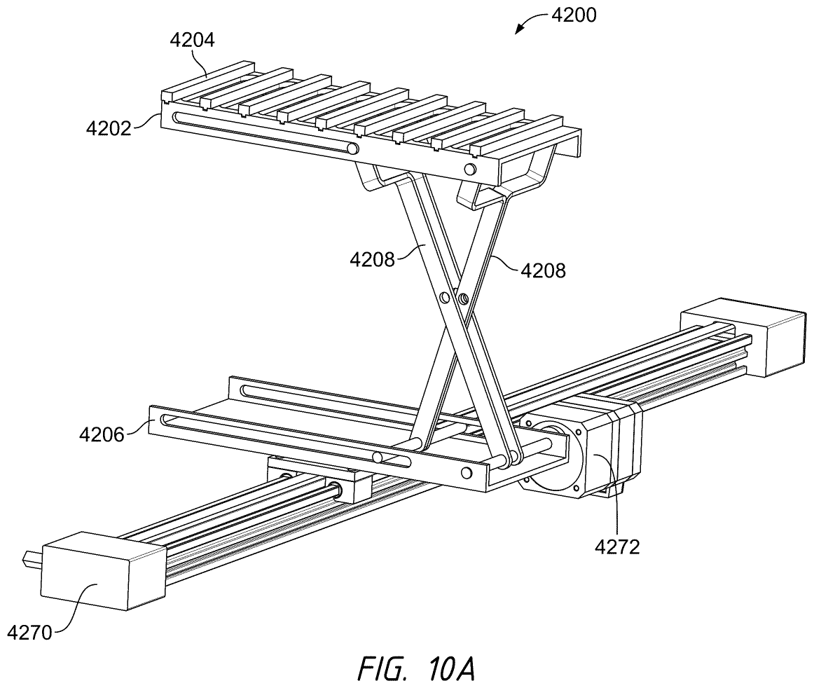

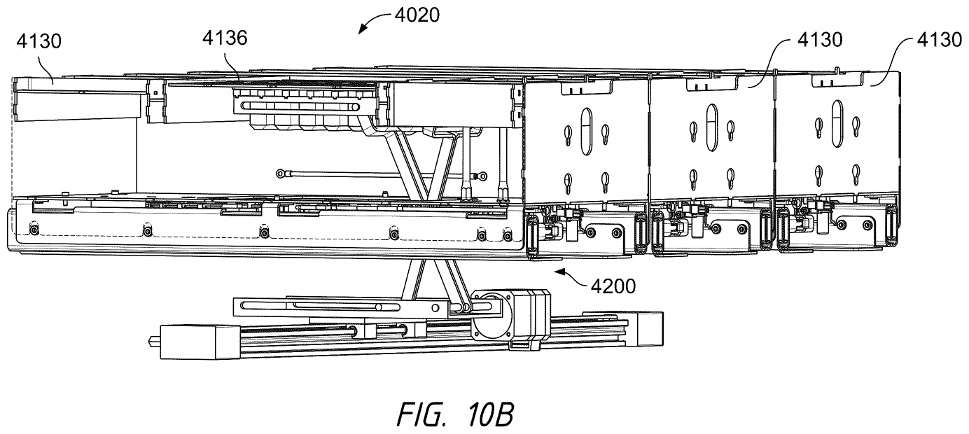

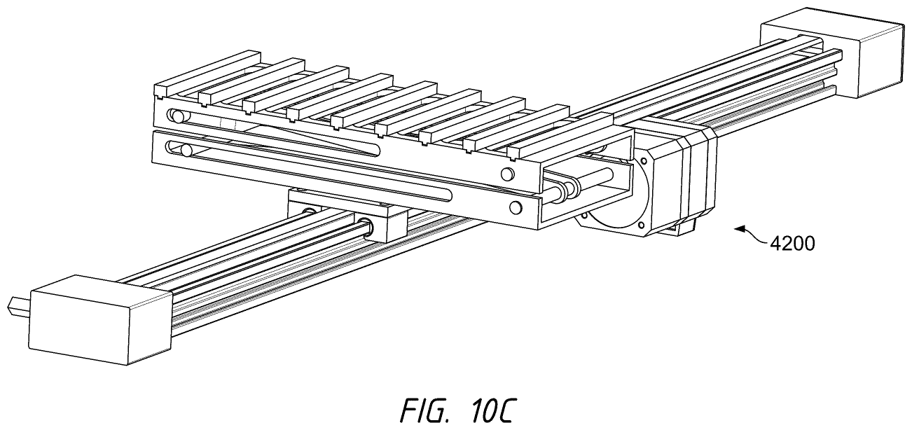

FIGS. 10A-10C are views of an extractor scissor lift according to the present disclosure.

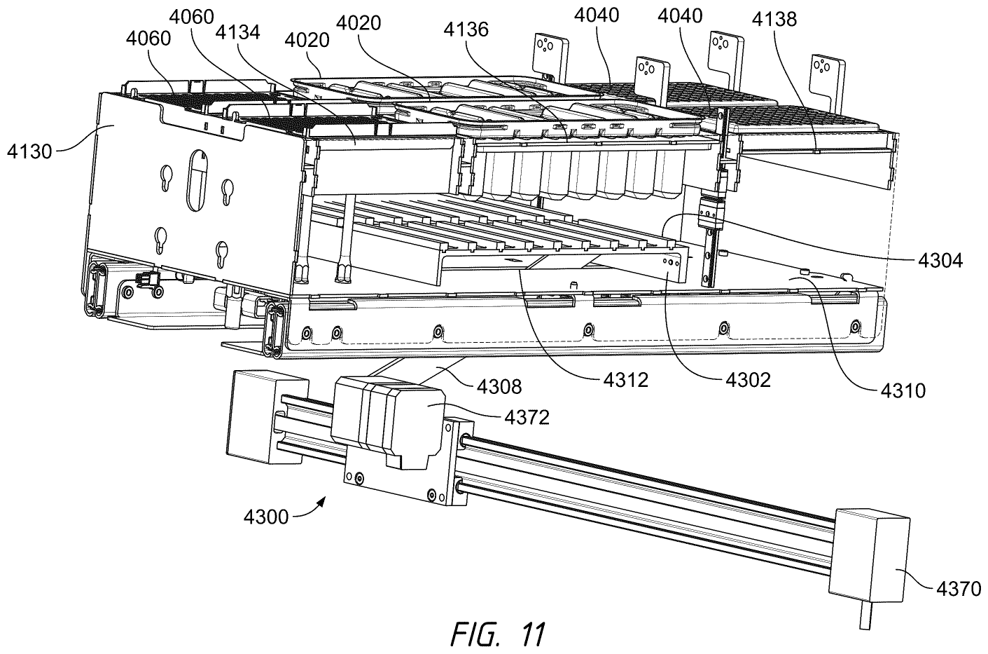

FIG. 11 is a view of an extractor pusher according to the present disclosure.

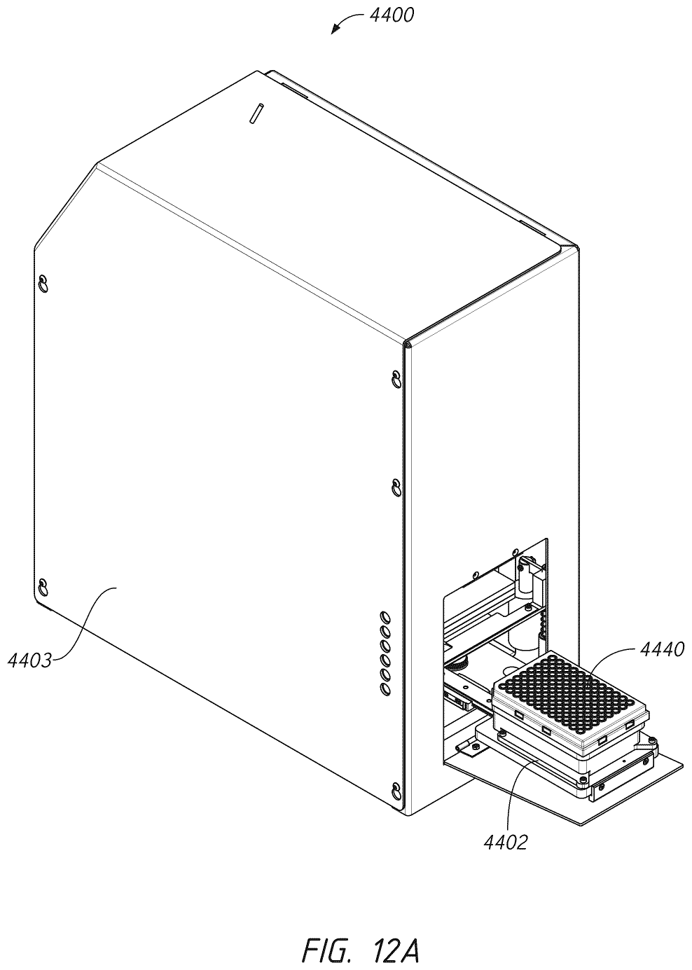

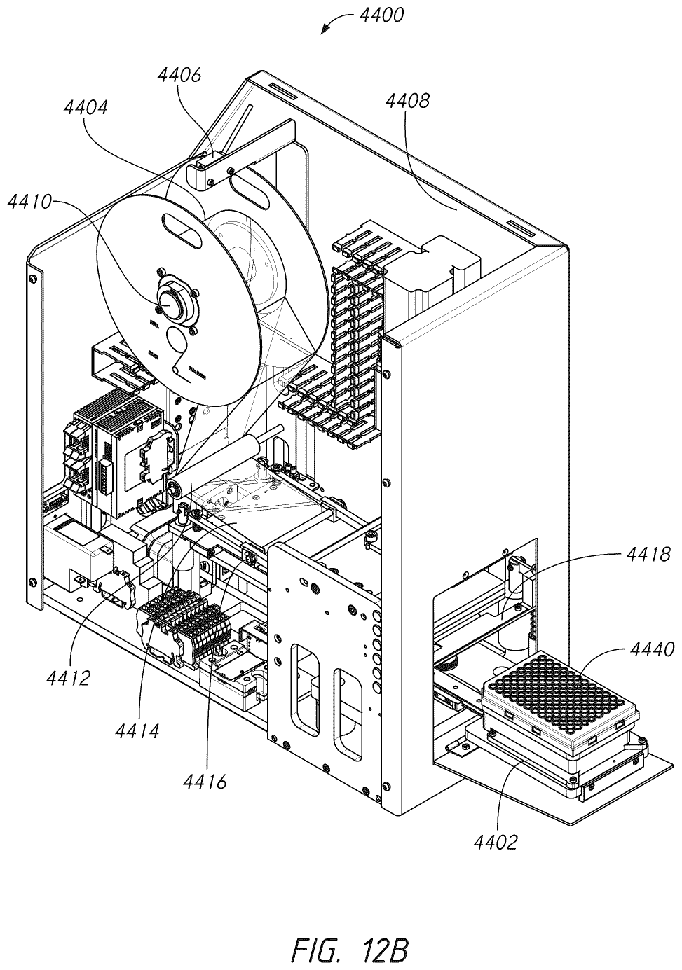

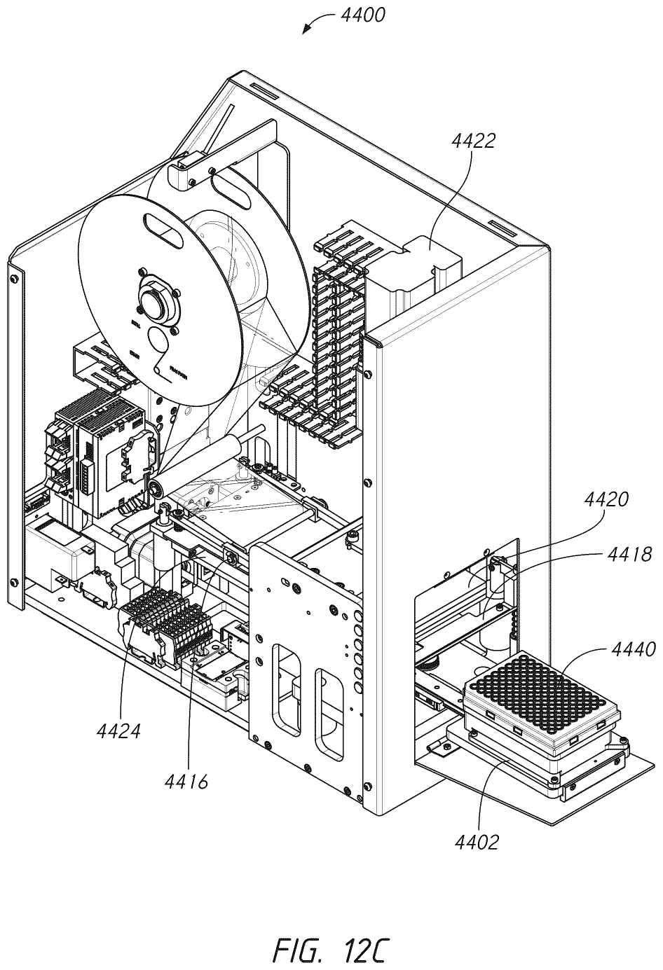

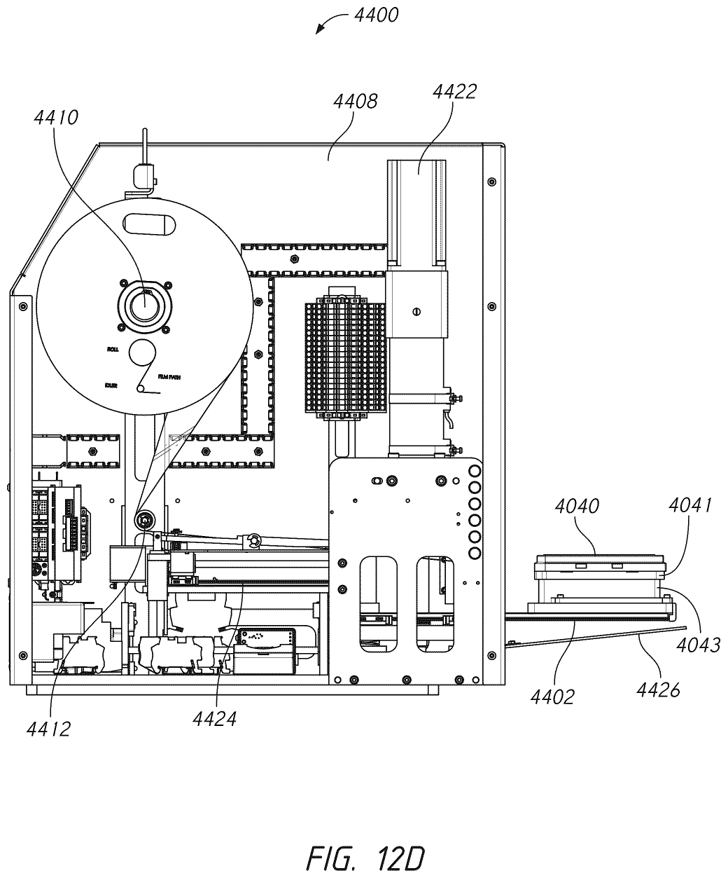

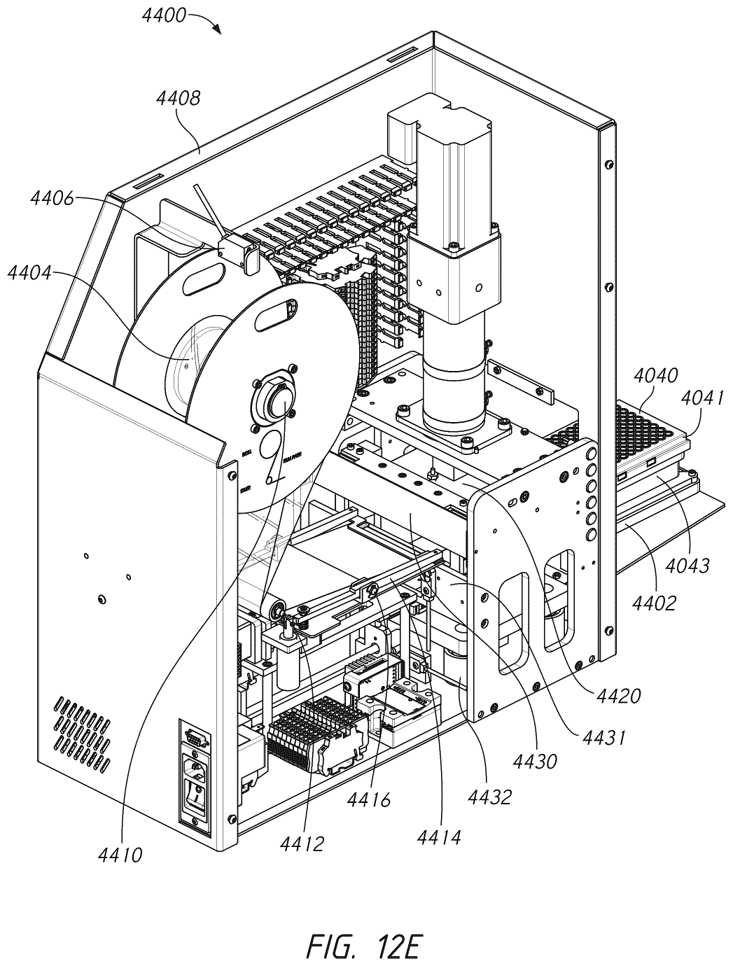

FIGS. 12A-12E are views of a plate sealer according to the present disclosure.

### DETAILED DESCRIPTION

Embodiments of the present disclosure provide devices, systems, and methods capable of processing a sample. In some embodiments, a multi-lane drawer is provided which hold consumables used during processing, such as two or more pipette tip holders, two or more extraction container holders, and two or more amplification plates. An embodiment includes one or more extractor modules configured to apply a magnetic field to extraction containers within the multi-lane drawer. In some embodiments, a plate sealer is provided which seals an amplification plate for processing. As described herein, the multi-lane drawer and the plate sealer can be components of a high-throughput system configured to process a plurality of a samples while reducing technician involvement. The systems described herein can prepare samples for analysis, while automating one or more steps between the receipt of the sample to producing test results.

FIG. 1 depicts a high-throughput system 00 according to an embodiment of the present disclosure. The high-throughput system 00 can include a first analyzer 2000. The high-throughput system 00 can include a second analyzer 4000. The high-throughput system 00 can include any number of analyzers. The high-throughput system 00 can include pre-analytical system 10. The analyzers 2000, 4000 and pre-analytical system 10 are modular such that they can be physically connected and disconnected from one another. The analyzers 2000, 4000 and pre-analytical system 10 are modular such that they can be electronically connected and disconnected from one another. In some embodiments, the analyzers 2000, 4000 can have the same configuration. The analyzers 2000, 4000 can have the same function. The analyzers 2000, 4000 can be duplicates of each other. The pre-analytical system 10 can couple to at least two of the same analyzers. In some embodiments, the analyzers 2000, 4000 can have a different configuration. The analyzers 2000, 4000 can have a different function. The analyzers 2000, 4000 can perform different operations. The analyzers 2000, 4000 can analyze different assays. The pre-analytical system 10 can couple to at least two different analyzers. The modularity of pre-analytical system 10 allows it to couple to any analyzer so configured. As shown, the first and second analyzers 2000, 4000 are disposed at opposite sides of pre-analytical system 10 in a linear arrangement. In other embodiments, the pre-analytical system 10 can be disposed at one end of the high-throughput system 00. The pre-analytical system 10 and analyzers 2000, 4000 can be arranged in any physical arrangement. The analyzer 4000 can be coupled to either side of pre-analytical system 10.

The pre-analytical system 10 can include a sample container shuttle transport assembly 4250, shown in FIG. 4A. The sample container shuttle transport assembly 4250 can transfer a sample container to one or more analyzers 2000, 4000. The sample container shuttle transport assembly 4250 of pre-analytical system 10 can extend toward analyzer 4000 when analyzer 4000 is located to the left of system 10 (exemplified in FIG. 1). In other embodiments, the sample container shuttle transport assembly 4250 of pre-analytical system 10 can extend toward analyzer 4000 where analyzer 4000 is located to the right of system 10. In some embodiments, the sample container shuttle transport assembly 4250 ends at the threshold of the analyzers 2000, 4000. In other embodiments, the sample container shuttle transport assembly 4250 extends into the analyzers 2000, 4000. The analyzer 2000, 4000 can have a conveyor that can continue the path of a respective shuttle transport assembly 4250 into analyzer 2000, 4000. The sample container shuttle transport assembly 4250 can be a rack or carrier structure with a plurality of receptacles, each receptacle sized and configured to receive a sample container. The sample container shuttle transport assembly 4250 can include an actuator which causes movement of the rack or carrier structure. The high-throughput system 00 can include an opening 120 to receive the rack or carrier structure with the plurality of sample containers. The analyzers 2000, 4000 can include one or more submodules 2110, 2120, 2130. These submodules can be the same or different from each other.

The high-throughput system 00 can include an overall system display interface 1332. Data can be entered and viewed through a graphical user interface (“GUI”) which may be displayed on the display interface 1332. Data can be entered and viewed through a graphical user interface which is specifically associated with analyzer 2000, 4000. Data can also be entered from a vision system of a multipurpose robot. Data can also be entered from scanners within pre-analytical system 10. Data can also be obtained by sensors, such as door sensors, to obtain information regarding certain conditions and activities occurring within analyzer 2000, 4000. Data can also be obtained by sensors, such as temperature sensors, to obtain information regarding certain conditions and activities occurring within analyzer 2000, 4000. Data can also be obtained about the location of one or more consumables. Data can also be obtained about the location of the sample container.

The pre-analytical system 10 can be an automated system for pre-analytical processing of sample to be assayed. The analyzer 2000, 4000 can be a modular system that is configured to operate in cooperation with the pre-analytical system 10. The analyzer 2000, 4000 can be configured for modular connectivity to the pre-analytical system 10. The analyzer 2000, 4000 can be configured for high-throughput processing and analysis of samples. The analyzer 4000 is described in greater detail below. The analyzer 2000 can include any of the features described herein.

FIGS. 2 and 3 show the analyzer 4000. The analyzer 4000 can include a structural frame 4011 comprised of several support components. The support components can be segments of metal tubing, as an example. The frame 4011 is configured to support and define various decks or levels for sample processing and analysis. The decks can include a supplementary deck 4012. The decks can include a processing deck 4014. The decks can include a multipurpose robot deck 4016. The analyzer 4000 can include a housing or shell that surrounds the frame 4011. The housing is shown in FIG. 1. The analyzer 4000 can include two detectors 4260a, 4260b. The detectors 4260a, 4260b can be located at opposite ends of analyzer 4000. The detectors 4260a, 4260b can have cavities that face the center of analyzer 4000. The detectors 4260a, 4260b can have a housing that receives a sealed amplification plate 4040, shown in FIG. 4F. The detectors 4260a, 4260b can have a thermocycler that is used to amplify a target analyte within the sealed amplification plate 4040. The detectors 4260a, 4260b can detect the target analyte using a set of illuminators, for example LED lights. The analyzer 4000 can include a plate sealer 4220. The plate sealer 4220 can seal the amplification plate 4040. The analyzer 4000 can include waste 4004 to dispose of the amplification plate 404. The analyzer 4000 can include a liquid waste repository that receives all liquid waste of the analyzer 4000. The analyzer can include a multipurpose robot 4300.

FIGS. 4A and 4B depict the processing deck 4014. The detectors 4260a, 4260b are shown in relation to the processing deck 4014. The sample container shuttle transport assembly 4250 is also shown, which can transport sample containers into the analyzer 4000. The processing deck 4014 can include one or more consumable drawers 4120. In the illustrated embodiments, six consumable drawers 4120 are shown. The processing deck 4014 can include any number of drawers including one, two, three, four, five, six, seven, eight, nine, ten, eleven, twelve, or any range of two of the foregoing values. Each drawer 4120 includes a single lane of consumables. Each drawer 4120 can hold a pipette tip holder 4060. Each drawer 4120 can hold an extraction container 4020. Each drawer 4120 can hold an amplification plate 4040. In the embodiment depicted, the processing deck 4014 includes six consumable drawer assemblies 4120. Each drawer 4120 can house the majority of the consumables utilized in an assay workflow.

The processing deck 4014 can include the plate sealer 4220. The plate sealer 4220 can have a moveable platform 4224 that receives an amplification plate 4040 and moves the amplification plate 4040 into the plate sealer 4220. The processing deck 4014 can include one or more orbital shakers 4230. The orbital shakers 4230 can oscillate one or more sealed amplification plates 4040 in a circular motion to fully rehydrate a dried reagent with an eluted sample within the compartments of the sealed amplification plate 4040. The processing deck 4014 can include reagent trough assemblies 4050. The reagent trough assembly 4050 can include troughs that house bulk reagents. The processing deck 4014 can include one or more piercing tools 4240. The piercing tool 4240 can be shaped to puncture the heavy duty seal of the reagent trough assembly 4050. The piercing tool 4240 sits within respective nests or carriers 4270, 4280 toward the center rear of processing deck 4014 until they are used to puncture a reagent trough assembly 4050. The processing deck 4014 can include one or more waste chutes 4210. The waste chutes 4210 can extend through processing deck 4014. The waste chutes 4210 can receive used amplification plates 4040. The processing deck 4014 can include a tip drawer 4110. The tip drawer 4110 can house one or more pipette tip holders 4060. In the illustrated embodiments, the tip drawer 4110 can hold five pipette tip holders 4060.

FIG. 4C depicts a side perspective view of the drawer 4120. The drawer 4120 can include a housing 4122. The drawer 4120 can include a pipette tip station 4124. The drawer 4120 can include an extraction container station 4126. The drawer 4120 can include an amplification plate station 4128. The stations 4124, 4126, 4128 can be linearly arranged. The pipette tip station 4124 can be near the front of the drawer 4120. The extraction container station 4126 can be between the pipette tip station 4124 and the amplification plate station 4128. The amplification plate station 4128 can be near the rear of the drawer 4120. The drawer 4120 can include other arrangements of the pipette tip station 4124, the extraction container station 4126, and the amplification plate station 4128. The pipette tip station 4124 can be configured to hold the pipette tip holder 4060. The extraction container station 4126 can be configured to hold the extraction container holder 4020. The amplification plate station 4128 can be configured to hold the amplification plate 4040. The drawer 4120 can be considered a one lane drawer. The drawer 4120 can hold a single pipette tip holder 4060. The drawer 4120 can hold a single extraction container holder 4020. The drawer 4120 can hold a single amplification plate 4040.

The drawer 4120 can be configured to include an extractor module 4125. The extractor module 4125 can be disposed within the housing 4122 of the drawer 4120. The extractor module 4125 can include one or more magnets. The magnets can be mounted to a plate or platform of the extractor module 4125. The magnets can be mounted in horizontal rows. The magnets can be disposed on either side of the extraction containers within the extraction container holder 4020. The extractor module 4125 can provide a movable magnetic field that is utilized to extract polynucleotides, such as DNA, from the samples. The extractor module 4125 can be housed in the consumable drawer 4120 beneath extractor container station 4126. The extractor module 4125 can be selectively moveable in an up-down direction along one or more rails 4127. The rails 4127 can be disposed on one or more sidewalls separating compartments beneath each of stations 4124, 4126 and 4128. The extractor module 4125 is in an up or extraction position in FIG. 4C. The extractor module 4125 can be lowered by movement along the rails 4127.

The drawer 4120 can include a hinged retention feature 4121. The retention feature 4121 can rotate about a hinge 4123. The retention feature 4121 can be a spring-loaded arm. The retention feature 4121 can be hingedly connected to housing 4122. The retention feature 4121 can be connected immediately behind the extraction container station 4126. The retention feature 4121 can have a retention position and consumable replacement position. In the retention position, as shown in FIG. 4C, the retention feature 4121 extends over the pipette tip station 4124 and the extraction container station 4126. The retention feature 4121 can include an opening allowing access to the pipettes within the pipette tip holder 4060. The retention feature 4121 can include an opening allowing access to the extraction containers within the extraction container holder 4020. In this position, the retention feature 4121 is configured to encompass the perimeter of the pipette tip holder 4060 located in the pipette tip station 4124. In this position, the retention feature 4121 is configured to encompass the perimeter of the extraction container holder 4020 located in the extraction container station 4126. The retention feature 4121 encompasses the perimeters while allowing access thereto via openings in retention feature 4121. The retention feature 4121 secures the edges of the pipette tip holder 4060. The retention feature 4121 secures the edges of the extraction container holder 4020. The retention feature 4121 limits or prevents the extraction container holder 4020 and the pipette tip holder 4060 from being inadvertently moved during operation. The retention feature 4121 limits or prevents liftoff during pipetting operations. When the consumables in the drawer 4120 need to be replaced, the drawer 4120 can be extended from the front of the analyzer 4000. In some embodiments, a feature configured to lock the retention feature 4121 in the retention position is released when the drawer 4120 is extended. Under the bias of a torsion spring located within hinge 4123, the retention feature 4121 rotates about hinge 4123 to the consumable replacement position which provides clearance for a user to replenish consumables within the drawer 4120.

Referring back to FIG. 4A, the drawers 4120 sit at the front of analyzer 4000. The drawers 4120 can be disposed between the detectors 4260a, 4260b. The drawers 4120 can include a visual indicator, such as a colored LED, on a front end thereof that indicates status of the drawer 4120 to a user. The visual indicator can let a user know that the drawer 4120 is currently being used. The visual indicator can let a user know that the drawer 4120 is ready to be used. The visual indicator can let a user know that the drawer 4120 needs replenishing with consumables.

FIGS. 4D-4E show the extraction container holder 4020. The extraction container holder 4020 can be a multi-layer structure. The extraction container holder 4020 can include a lower portion 4025 with a plurality of openings 4029. The lower portion 4025 can include one or more features 4028. The features 4028 on sidewalls 4027 of the lower portion 4025 can provide an interference fit with features on the drawer 4120 of analyzer 4000. The extraction container holder 4020 can include a plurality of extraction containers 4026. The extraction containers 4026 can be connected via one or more foil strips 4023. The foil strip 4023 is penetrable with a pipette tip prior to the addition of a sample. Each extraction container 4026 may contain Ferric Oxide (“FOX”) particles to extract polynucleotides such as DNA from samples. The extraction container holder 4020 can include an upper portion 4022 with a plurality of openings 4021. The strip 4023 can be aligned within the openings 4021. The upper portion 4022 can include one or more ribs 4024. The ribs 4024 can extend in a direction transverse to the foil strips 4023 of the extraction containers 4026. The ribs 4024 can provide structural stiffness to extraction container holder 4020. The lower portion 4025 allows the extraction containers 4026 to partially extend there through. The extraction containers 4026 extend through the extraction container holder 4020 to allow processing by the extractor module 4125. A barcode can be located on upper portion 4022 which helps track information such as lot, expiration date, and serial number of the contents of the extraction containers 4026. The extraction container holder 4020 can be assembled with enough extraction containers 4026 to perform a single run. In the embodiment depicted, the extraction container holder 4020 can house 32 extraction containers 4026. The extraction containers 4026 can be arranged along four foil strips 4023. The extraction containers 4026 can be arranged in a grid.

The pipette tips are provided in the pipette tip holder 4060. The pipette tip holder 4060 can be configured to house 96 pipette tips. The pipette tips can be arranged in a grid. In some embodiments of the analyzer 4000, four 1000-μL tips are used to process each sample. In addition, a single reagent pipette tip can be used with each batch of samples. This helps reduce the number of tips utilized as the reagent pipette tip does not come into direct contact with samples.

FIG. 4F shows the amplification plate 4040. The amplification plate 4040 can include a plate body 4041. The plate body 4041 can include one or more engagement openings 4044a, 4044b that extend into respective sides 4042a, 4042b of the body 4041. The engagement openings 4044a, 4044b can allow a gripper of the multipurpose robot 4300 to engage amplification plate assembly 4040 from opposing sides thereof. For example, the openings 4044a extend through the side 4042a and a side directly opposite that of the side 4042a. In addition, the openings 4044b extend through the side 4042b and through a side directly opposite that of the side 4042b. These openings allow the multipurpose robot 4300 to grip and lift the amplification plate 4040 while the amplification plate 4040 is in different orientations. The plate body 4041 can include a plurality of wells that define amplification compartments 4045. The compartments 4054 can be provided with dried reagents that are utilized for amplification of a target. The amplification plate 4040 can have a visual identification of the reagents contained in compartments 4045 of the amplification plate 4040, such as a color coding.

When each drawer 4120 is extend from the analyzer 4000, the user removes and replaces the used amplification container holders 4020 and empty tip holders 4060. The user also adds an unused amplification plate 4040 to the drawer 4120. Once drawer 4120 is reinserted, the instrument 00 can inventory that particular drawer 4120 to check for loading errors. The instrument 00 can be updated to indicate the drawer 4120 as ready for an extraction.

Referring back to FIG. 4A, the processing deck 4014 also includes the tip drawer 4110. The tip drawer 4110 can configured to hold one or more pipette tip holders 4060. The tip drawer 4110 can house five pipette tip holder 4060. Each pipette tip holder 4060 can be a 96-well tip carrier. The tip drawer 4110 can be similarly constructed to the drawers 4120 in that it includes visual indicators on a front end thereof. The pipette tip holders 4060 in the tip drawer 4110 can provide the pipette tip utilized for each sample extraction conducted in the consumable drawers 4120, along with reagent tips and any excess tips that may be needed due to pick-up failures or clogs. The tip drawer 4110 can be disposed to the left of the consumable drawers 4120.

These drawers 4110, 4120 can be accessed from the front of analyzer 4000 by a user. The access to the drawers 4110, 4120 can be automated. The tip drawer 4110 can be automatically locked or unlocked by analyzer 4000. The tip drawer 4110 can be locked or unlocked depending on the present status of the tip drawer 4110. The tip drawer 4110 can be locked or unlocked depending on the present status of the analyzer 4000. Each drawer 4120 can be automatically locked or unlocked by analyzer 4000. Each drawer 4120 can be locked or unlocked depending on the present status of the drawer 4110. Each drawer 4120 can be locked or unlocked depending on the present status of the analyzer 4000. The tip drawer 4110 and the one or more drawers 4120 can be locked and unlocked simultaneously. The tip drawer 4110 and the one or more drawers 4120 can be locked and unlocked independently. The drawer 4110, 4120 can include a latching feature that locks the drawer 4110, 4120 in the fully open position. The drawer 4110, 4120 can include a latching feature that locks the drawer 4110, 4120 in the fully closed position. The drawer 4110, 4120 can include a latching feature that locks the drawer 4110, 4120 in both the fully open position and the fully closed position. The latching mechanism can be a push-to-close latch. The latching mechanism can be fully closed by a simple push or other action of the user. The latching mechanism can be spring loaded. The latching mechanism can hold the drawer 4110, 4120 securely within the analyzer 4000. The latching mechanism can be built-in. The latching mechanism can include a trigger that is biased toward a locking configuration. The trigger can be depressed to unlock the drawer. The trigger can be depressed automatically by a module of the analyzer 400. The trigger can be depressed by a user. The drawer 4120 can be considered a single lane drawer. The drawer 4120 can include one latching mechanism to lock and unlock the drawer 4120.

Referring back to FIG. 2, the supplementary deck 4012 is disposed adjacent the bottom of analyzer 4000 and is located beneath processing deck 4014. The supplementary deck 4012 houses electronic components and waste repositories. For example, supplementary deck 4012 can include the liquid waste repository 4002 that receives and houses all liquid waste, such as from the extraction containers 4026 during extraction processes and from the trough assembly 4050 during an emptying process. The supplementary deck 4012 also includes one or more waste repositories 4004 that sit below each of waste chutes 4210 that extend through processing deck 4014, as shown in FIG. 4A. For example, a single waste repository 4004 may be located under waste chutes 4210 and may collect all solid waste. In another example, two waste repositories 4004 may be used to collect used pipette tips and used amplification plates 4040, respectively. Each waste repository 4004 may contain a sensing apparatus for detecting waste level. The sensing apparatus can include an optical or ultrasonic sensor, for example.

In the embodiment depicted, the processing deck 4014 includes six consumable drawer assemblies 4120. Each drawer assembly 4120 is a single lane drawer. There are a total of six drawers 4120. In some cases, this drawer arrangement increases cost and there is a need to reduce costs. In some cases, this drawer arrangement is complex to manufacture or service and there is a need to reduce complexity in manufacturing and service. For example, each drawer 4120 includes its own extractor module 4125. For another example, each drawer 4120 includes its own latching feature. There is a need to reduce components and thus costs related to the drawer assemblies. The drawers described herein can include any of the features of the drawer 4120.

FIGS. 5A-5C are views of an embodiment of a two lane drawer 4130 and corresponding extractor module 4135 according to the present disclosure. The two lane drawer 4130 can include any of the features of the drawer 4120. The two lane drawer 4130 can include advantageous features. The two lane drawer 4130 can be more cost effective. The two lane drawer 4130 can hold the same number of consumables as two of the drawers 4120. The two lane drawer 4130 can advantageously have only one extractor module 4135. The two lane drawer 4130 can advantageously have only one set of telescoping slides to facilitate the movement of the two lane drawer 4130. The two lane drawer 4130 can advantageously have only one indicator related to the use of the two lane drawer 4130. The two lane drawer 4130 can advantageously have only one latching feature that prevents withdrawal of the two lane drawer 4130 during use. The two lane drawer 4130 can advantageously have only one retention feature to retain the consumables within the two lane drawer 4130. Referring back to FIG. 4A, the analyzer 4000 can include six lanes of consumables with six drawers 4120, six corresponding extractor modules 4125, and six corresponding latching mechanisms. With the two lane drawer 4130, the same analyzer 4000 can hold six lanes of consumables but with only three of the two lane drawers 4130, three extractor modules 4135, and three corresponding latching mechanisms. The two lane drawer 4130 can reduce the number of parts. The two lane drawer 4130 can reduce costs. The two lane drawer 4130 can be reduce complexity in manufacturing and servicing. The two lane drawer 4130 does not impact total throughput since the number of consumables is the same, and thus the number of samples processed is the same. The two lane drawer 4130 does not impact customer walk-away time since the number of consumables is the same, and thus the number of samples processed is the same.

The two lane drawer 4130 is configured to house the majority of the consumables utilized in an assay workflow. The two lane drawer 4130 can include a pipette tip station 4134. The two lane drawer 4130 can include an extraction container station 4136. The two lane drawer 4130 can include an amplification plate station 4138. The pipette tip station 4134 is configured to hold two pipette tip holders 4060. The extraction container station 4136 is configured to hold two extraction container holders 4020. The amplification plate station 4138 is configured to hold two amplification plates 4040. The two lane drawer 4130 can be configured to hold the same types of consumables described herein. The two lane drawer 4130 can hold twice the number of consumables as the drawer 4120.

The stations 4134, 4136, 4138 can be arranged linearly. The arrangement can be from front to back, the pipette tip station 4134 in the front, the extraction container station 4136 in the middle, and the amplification plate station 4138 in the back. The two lane drawer 4130 can hold two pipette tip holders 4060 side-by-side in the front. The two lane drawer 4130 can hold two extraction container holders 4020 side-by-side in the middle. The two lane drawer 4130 can hold two amplification plates 4040 side-by-side in the back.

The arrangement can allow the consumables to interface with other components of the analyzer 4000. The analyzer 4000 can include three of the two lane drawers 4130. The two lane drawers 4130 can sit at the front of analyzer 4000 between two detector 4260a, 4260b. The extraction container station 4136 can facilitate the extraction of polynucleotides from samples. The extraction container station 4136 can be located in relation to the extractor module 4135. Once extraction is finished, the extracted polynucleotides in the extraction containers 4026 are used to rehydrate a reagent, for example a master mix reagent, in one of the amplification plates 4040 in the amplification plate station 4138. The amplification plates 4040 can be prepared for amplification, such as PCR. The prepared amplification plates 4040 can be moved to the appropriate reader 4260a, 4260b. This process can be achieved through use of multifunctional robot 4300, the plate sealer 4220, and orbital mixer 4230. The two lane drawer 4130 can include a visual indicator that indicates the current status of the two lane drawer 4130.

The two lane drawer 4130 houses the extractor module 4135 within a housing 4132. The two lane drawer 4130 can include one extractor module 4135. In some embodiments, the extractor module 4135 can process samples from two lanes simultaneously. In some embodiments, the extractor module 4135 can process samples from two extraction container holders 4020 simultaneously. In some embodiments, the extractor module 4135 can process twice the number of samples as the extractor module 4125. The extractor module 4135 can move up and down relative to two extraction container holders 4020 simultaneously. In some embodiments, the extractor module 4135 can process samples from two lanes sequentially, for instance, depending on when samples are inserted into the extraction containers 4026. In some embodiments, the extractor module 4135 can process samples from two extraction container holders 4020 sequentially. In some embodiments, the extractor module 4135 can process samples from two lanes independently, for instance, depending on when samples are inserted into the extraction containers 4026. In some embodiments, the extractor module 4135 can process samples from two extraction container holders 4020) independently. In some embodiments, pipettors are only extracting polynucleotides from one of the extraction container holders 4020. In some embodiments, the workflow does not process samples from two extraction container holders 4020. The software can be limited to processing one extraction container holder 4020. The pipettors can be limited to processing one extraction container holder 4020. The instrument can be limited to processing one extraction container holder 4020. The workflow timing can be limited to processing one extraction container holder 4020.

The extractor module 4135 includes magnets 4136. The magnets 4136 can be arranged to apply a magnetic field on opposite sides of extraction containers. The magnets 4136 provide the magnetic field that is utilized to extract polynucleotides from the samples. The extractor module 4135 is movable vertically. The extractor module 4135 can move the magnets 4136, and thus the corresponding magnetic field relative to the extraction containers 4026 of the two extraction container holders 4020 located in the extraction container station 4136. The extractor module 4135 can be positioned beneath the extractor container station 4136. The extractor module 4135 can be selectively moveable in an up-down direction along one or more rails 4137. The rail 4137 can be disposed on a sidewall separating the extraction container station 4136 and the amplification plate station 4138. As depicted in FIG. 5C, the extractor module 4135 is in an extended position. The magnets 4136 can be near the extraction containers 4026. The extractor module 4135 can be lowered to be below the extraction containers 4026. The extractor module 4135 is housed in the two lane drawer 4130. In some embodiments, the analyzer 4000 can include three of the two lane drawers 4130 and three extractor modules 4135.

This movement of magnets 4136 of the extractor module 4135 can facilitate processing of samples. To help isolate the polynucleotides that are extracted from the sample, the polynucleotides can bound to ferric oxide (“FOX”) particles located within the extraction containers 4026. The particles allow for the magnetic capture of the polynucleotides. This enables the polynucleotides to be isolated from the rest of the unwanted sample, which can be washed away from the eluate using a wash buffer located in the trough assembly 4050. In order to perform this isolation, a magnetic field is applied to the extraction containers 4026 of the two extraction container holders 4020. The magnetic field is applied through the use of the extractor module 4135. The extractor module 4135 includes magnets 4136 positioned to ensure that each row of extraction containers 4026 is subjected to the magnetic field. In some embodiments, magnets 4136 are disposed on both sides of the extraction container 4026. The magnets 4136 are selectively moved from a position entirely below the extraction containers 4026 to a position adjacent to the extraction containers 4026. In some embodiments, the magnets 4136 can generally lie in a plane. The extraction containers 4026 can intersect that plane in an extraction mode. The extraction containers 4026 can be entirely below the plane in other modes. In some embodiments, the magnets 4136 overlap with the extraction containers 4026. The extractor module 4135 applies the magnetic field which captures the bound polynucleotides to a side of the extraction containers 4026.

The two lane drawer 4130 can include a hinged retention feature 4131. The retention feature 4131 can be a spring loaded plate. The retention feature 4131 can be hingedly connected to the housing 4132. The hinge 4133 of the retention feature 4131 can be positioned behind extraction container station 4136. The hinge 4133 of the retention feature 4131 can be positioned in front of the amplification plate station 4138. The retention feature 4131 has a retention position and consumable replacement position. In the retention position, as shown in FIG. 5A, the retention feature 4131 extends over the pipette tip station 4134 and the extraction container station 4136. In this position, the retention feature 4131 is configured to extend over at least a portion of the two pipette tip holders 4060 and the two extraction container holders 4020 that are located in the pipette tip station 4134 and the extraction container station 4136, respectively. The retention feature 4131 can cover the perimeter of the two pipette tip holders 4060 and the perimeter the two extraction container holders 4020 while allowing access thereto via openings in retention feature 4131. The retention feature 4131 functions as a cover to limit movement of the two extraction container holders 4020 and the two pipette tip holders 4060 during operation, such as pipetting operations. The hinged retention feature 4131 can include a locking feature that locks retention feature 4131 in the retention position.

When consumables in the two lane drawer 4130 need to be removed and replaced with new consumables, the two lane drawer 4130 can be extended. The locking feature that locks retention feature 4131 in the retention position can be released upon extension of the two lane drawer 4130. The locking feature that locks retention feature 4131 in the retention position can be a magnet. When the two lane drawer 4130 is open, the retention feature 4131 can also be lifted. The retention feature 4131 is not dependent on the extension of the two lane drawer 4130. Under the bias of a torsion spring, the retention feature 4131 can rotate about the hinge 4133 to the consumable replacement position. The consumable replacement position opens the retention feature 4131 to provide clearance for a user to replenish consumables within the two lane drawer 4130. The two lane drawer 4130 can be extended along telescoping slides. The two lane drawer 4130 can include an assembly of extensions that slide relative to each other. The slides can be metal. The slides can include ball bearings. The slides can be full extension slides. The slides can extend in sections. The slides can extend in stages. The housing 4132 of the two lane drawer 4130 can include tab and slot weldments. The tab and slot arrangement can join one surface to another through an elongated hole.

The two lane drawer 4130 can include a latching feature that locks the two lane drawer 4130 to prevent extension of the two lane drawer 4130 from the analyzer 400. The two lane drawer 4130 can including a latching feature that locks the two lane drawer 4130 in the fully open position. The two lane drawer 4130 can including a latching feature that locks the two lane drawer 4130 in the fully closed position. The latching mechanism can be a push-to-close latch. In some embodiments, the latching mechanism does not lock in the open position. In some embodiments, the latching mechanism does not accommodate being locked in the open position. In some embodiments, the latching mechanism can be locked in the open position with different telescoping slides. The latching mechanism can be fully closed by a simple push or other action of the user. The latching mechanism can be spring loaded. The latching mechanism can hold the two lane drawer 4130 securely within the analyzer 4000. The latching mechanism can be built-in. The latching mechanism can include a trigger that is biased toward a locking configuration. The trigger can be depressed to unlock the drawer. The trigger can be depressed automatically by a module of the analyzer 4000. The trigger can be depressed by a user. The two lane drawer 4130 can include one latching mechanism to lock and unlock the two lane drawer 4130.

The two lane drawer 4130 can include a kick out mechanism. The kick-out mechanism can facilitate the sliding extension of the two lane drawer 4130. The kick-out mechanism can extend along the bottom of the two lane drawer 4130. The purpose of the kick-out mechanism can be to open the two lane drawer 4130 at least a short distance after the two lane drawer 4130 has been unlocked for easy access to the handle. The kick-out mechanism can open the two lane drawer 4130 by 0.5″, 1″, 1.5″, 2″, 2.5″, 3″, 3.5″, 4″, at least 0.5″, at least 1″, at least 1.5″, at least 2″, at least 2.5″, at least 3″, at least 3.5″, at least 4″, or any range of two of the foregoing values. In some embodiments, the kick-out mechanism can open the two lane drawer by at least two inches. The purpose of the kick-out mechanism can be to push the two lane drawer 4130 against the latch when locked for positional repeatability. In some embodiments, the kick-out mechanism is not able to release the latch mechanism. In some embodiments, the user does not interact with the kick-out mechanism. The kick-out mechanism can allow the two lane drawer 4130 to slide relative to the analyzer 4000.

FIGS. 6A-6C are view of the two lane drawer 4130 in relation to the analyzer 4000 according to the present disclosure. FIG. 6A is an example of the analyzer 4000. The analyzer 4000 can include three of the two lane drawers 4130. The two lane drawer 4130 can house consumables, as described herein. The analyzer 4000 can include the tip drawer 4110. The tip drawer 4110 can house pipette tip holders 4060, as described herein.

FIG. 6B is a view of analyzer 4000 which includes the structural frame 4011 which is configured to support and define various decks or levels for sample processing and analysis. The analyzer 4000 can include the supplementary deck 4012, the processing deck 4014, and the multipurpose robot deck 4016, as described herein. The analyzer 4000 can includes a housing or shell that surrounds the internal components, as shown in FIG. 6A.

FIGS. 6A-6C illustrate the analyzer 4000 including three of the two lane drawers 4130. The two lane drawers 4130 can be adjacent. The two lane drawers 4130 can slide relative to a single drawer plate 4140. The drawer plate 4140 can remain stationary as the two lane drawers 4130 pull out individually relative to the plate 4140. The two lane drawers 4130 can extend relative to the drawer plate 4140. Each of the two lane drawers 4130 can slide relative to the drawer plate 4140. Each of the two lane drawers 4130 can include telescoping slides to allow the two lane drawer 4130 to slide relative to the drawer plate 4140. Each two lane drawer 4130 can slide independently of another two lane drawer 4130. Each two lane drawer 4130 can slide sequentially with another two lane drawer 4130. Each two lane drawer 4130 can slide simultaneously with another two lane drawer 4130. Each two lane drawer 4130 can include a latching mechanism. Each two lane drawer 4130 can include a kick-out mechanism. Each two lane drawer 4130 can include the retention feature 4131 that extends over the corresponding pipette tip station 4134 and the extraction container station 4136. The two lane drawers 4130 can be identical. The two lane drawers 4130 can be stacked side-by-side. The two lane drawers 4130 can include tab and slot weldments.

FIG. 7 is a view of an embodiment of a two lane drawer 4150 according to the present disclosure. The two lane drawer 4150 can include any feature of the drawers described herein. The two lane drawer 4150 can be used in combination with the extractor module 4135. The two lane drawer 4150 can hold the same number of consumables as the two lane drawer 4130. The two lane drawer 4150 can hold the same number of consumables as two of the drawers 4120. The two lane drawer 4150 only has one extractor module, similar to the drawer 4130. The two lane drawer 4150 only has one latching feature, similar to the drawer 4130. This results in the ability to hold six lanes of consumables but with only three extractors. This results in the ability to hold six lanes of consumables but with only three latching features. The two lane drawer 4150 can reduce the number of parts. The two lane drawer 4150 can reduce costs. The two lane drawer 4150 can reduce complexity in manufacturing and servicing.

The two lane drawer 4150 is configured to house the majority of the consumables utilized in an assay workflow. The two lane drawer 4150 includes a pipette tip station 4154, an extraction container station 4156, and an amplification plate station 4158. The pipette tip station 4154 is configured to hold two pipette tip holders 4060. The extraction container station 4156 is configured to hold two extraction container holders 4020. The amplification plate station 4158 is configured to hold two amplification plates 4040.

The analyzer 4000 can include a stationary retention housing 4161. In the depicted embodiment, the stationary retention housing 4161 can be a cover positioned relative to the two lane drawer 4150. The stationary retention housing 4161 extends over the pipette tip station 4154 and the extraction container station 4156. In this position, the stationary retention housing 4161 is configured to extend over at least a portion of the two pipette tip holders 4060 and over at least a portion of the two extraction container holders 4020 that are located in the pipette tip station 4154 and the extraction container station 4156, respectively. The stationary retention housing 4161 can cover a perimeter of the two pipette tip holders 4060 and the two extraction container holders 4020 while allowing access thereto via openings in the stationary retention housing 4161. The stationary retention housing 4161 functions to limit movement of the extraction container holder 4020 and the pipette tip holder 4060 during operation. In some embodiments, the stationary retention housing 4161 does not cover the amplification plates 4040 located in the amplification plate station 4158. The stationary retention housing 4161 can be fixed relative to a drawer plate 4160. The two lane drawer 4150 can extend relative to the drawer plate 4160.

When consumables in drawer 4150 need to be removed and replaced with new consumables, the two lane drawer 4150 is extended while the stationary retention housing 4161 does not move relative to the two lane drawer 4150. The two lane drawer 4150 slides relative to the stationary retention housing 4161. The two lane drawer 4150 can include a latching feature. The two lane drawer 4150 can include a kick-out mechanism. The two lane drawer 4150 can extended along telescoping slides. The stationary retention housing 4161 can include sheet metal with tab and slot weldments.

FIG. 8 is a view of an embodiment of a three lane drawer 4170 according to the present disclosure. The three lane drawer 4170 can include any feature of the drawers described herein. The three lane drawer 4170 can be used in combination with an extractor module. The extractor module can move relate to the three lanes. In some embodiments, the extractor module can apply a magnetic field to three extraction container holders 4020. The three lane drawer 4170 can hold the same number of consumables as three of the drawers 4120. The three lane drawer 4170 can have only one extractor module. The three lane drawer 4170 can have only one latching feature. This results in the ability to hold six lanes of consumables but with only two extractors and two latching mechanisms. The three lane drawer 4170 can reduce the number of parts. The three lane drawer 4170 can reduce costs. The three lane drawer 4170 can reduce complexity in manufacturing and servicing.

The three lane drawer 4170 is configured to house the majority of the consumables utilized in an assay workflow. The three lane drawer 4170 includes a pipette tip station 4174, an extraction container station 4176, and an amplification plate station 4178. The pipette tip station 4174 is configured to hold three pipette tip holders 4060. The extraction container station 4176 is configured to hold three extraction container holders 4020. The three extraction container holders 4020 can be positioned side-by-side. The amplification plate station 4178 is configured to hold three amplification plates 4040.

The analyzer 4000 can include a stationary retention housing 4181. The stationary retention housing 4181 can be a stationary cover positioned relative to the three lane drawer 4170. The stationary retention housing 4181 extends over the pipette tip station 4174 and the extraction container station 4176. In this position, the stationary retention housing 4181 is configured to extend over at least a portion of the three pipette tip holders 4060 and at least a portion of the three extraction container holders 4020 that are located in the pipette tip station 4174 and the extraction container station 4176, respectively. The stationary retention housing 4181 can cover the perimeters of the pipette tip holders 4060 and the perimeters of the extraction container holders 4020 while allowing access thereto via openings in the stationary retention housing 4181. The stationary retention housing 4181 functions to limit movement of the extraction container holders 4020 and the pipette tip holders 4060 during operation. In some embodiments, the stationary retention housing 4181 does not cover the amplification plates 4040 located in the amplification plate station 4178.

When consumables in three lane drawer 4170 need to be removed and replaced with new consumables, the three lane drawer 4170 is extended while the stationary retention housing 4181 does not move relative to the three lane drawer 4170. The three lane drawer 4170 can include a latching feature. The three lane drawer 4170 can include a kick-out mechanism. The three lane drawer 4170 can extended along telescoping slides. The stationary retention housing 4181 can include sheet metal with tab and slot weldments.

FIGS. 9A-9B are views an embodiment of a six lane drawer 4190 according to the present disclosure. The six lane drawer 4190 can be used in combination with the tip drawer 4110. The tip drawer 4110 can be configured to hold multiple pipette tip holders 4060. The tip drawer 4110 can house five pipette tip holders 4060. The six lane drawer 4190 and the tip drawer 4110 can be arranged side by side. In some embodiments, the six lane drawer 4190 and the tip drawer 4110 can be unitary. The six lane drawer 4190 and the tip drawer 4110 can move together to extend from the analyzer 4000.

The six lane drawer 4190 can include any feature of the drawers described herein. The six lane drawer 4190 can be used in combination with an extractor module. The extractor module can move relate to six lanes. The extractor module can apply a magnetic field to one extraction container holder 4020. The extractor module can apply a magnetic field to two or three extraction container holders 4020. The extractor module can apply a magnetic field to six extraction container holders 4020. The extractor module can move along an axis relative to the six lane drawer 4190. The extractor module can include a slide 4270. The slide 4270 can be a rail along which the extractor module slides. The slide 4270 is described in more detail below with reference to FIG. 10A. The six extraction container holders 4020 can be positioned side-by-side. The six lane drawer 4190 can hold the same number of consumables as six of the drawers 4120. The six lane drawer 4190 can have only one extractor module. The six lane drawer 4190 can have only one latching feature. This results in the ability to hold six lanes of consumables but with only one extractor module and one latching mechanism. The six lane drawer 4190 can reduce the number of parts. The six lane drawer 4190 can reduce costs. The six lane drawer 4190 can reduce complexity in manufacturing and servicing.

The six lane drawer 4190 is configured to house the majority of the consumables utilized in an assay workflow. The six lane drawer 4190 includes a pipette tip station 4194, an extraction container station 4196, and an amplification plate station 4198. The pipette tip station 4194 is configured to hold six pipette tip holders 4060. The extraction container station 4196 is configured to hold six extraction container holders 4020. The amplification plate station 4198 is configured to hold six amplification plates 4040.

The analyzer 4000 can include a stationary retention housing 4115. The stationary retention housing 4115 can be a single unitary structure that extends over the tip drawer 4110, at least a portion of the pipette tip station 4191, and at least a portion of the extraction container station 4196. The stationary retention housing 4115 can be a stationary cover positioned relative to the tip drawer 4110. The stationary retention housing 4115 can be a stationary cover positioned relative to the six lane drawer 4190. The stationary retention housing 4115 can extend over the pipette tip station 4194 and the extraction container station 4196. In this position, the stationary retention housing 4115 is configured to extend over at least a portion of the six pipette tip holders 4060 that are located in the pipette tip station 4194 of the six lane drawer 4190. In this position, the stationary retention housing 4115 is configured to extend over at least a portion of the five pipette tip holders 4060 of the tip drawer 4110. In this position, the stationary retention housing 4115 is configured to extend over at least a portion of the six extraction container holders 4020 that are located in the extraction container station 4196, respectively. The stationary retention housing 4115 can be a single unitary structure that retains consumables within the tip drawer 4110 and the six lane drawer 4190.

The stationary retention housing 4115 can cover the perimeters of the pipette tip holders 4060 and the perimeters of the extraction container holders 4020 while allowing access thereto via openings in the stationary retention housing 4115. The stationary retention housing 4115 functions to limit movement of the extraction container holders 4020 and the pipette tip holders 4060 during operation. In some embodiments, the stationary retention housing 4115 does not cover the amplification plates 4040 located in the amplification plate station 4198. The stationary retention housing 4115 can include a section for the tip drawer 4110. The stationary retention housing 4115 can include sheet metal with tab and slot weldments. The stationary retention housing 4115 can include a separate top plate mounted on the outside of supports. The stationary retention housing 4115 can include supports mounted to the sides of a base 4116. The stationary retention housing 4115 can include vertical supports to resist warping.

When consumables in the six lane drawer 4190 need to be removed and replaced with new consumables, the six lane drawer 4190 is extended while the stationary retention housing mechanism 4115 does not move relative to the six lane drawer 4190. The six lane drawer 4190 can include a latching feature. The six lane drawer 4190 can include a kick-out mechanism. The six lane drawer 4190 can extend along telescoping slides. In some embodiments, each lane of the six lane drawer 4190 slides together. The six lane drawer 4190 can include a single drawer front. In an alternate embodiment that is not illustrated, each lane of the six lane drawer 4190 can slide individually when each lane is associated with a respective one of a plurality of drawer fronts. Each lane can include an individual sliding sub drawer. The six lane drawer 4190 can be associated with an extractor having any of the features described herein. In some embodiments, when a run is in process on a lane, the extractor can move up and lock the lane so that the lane cannot be opened.

FIGS. 10A-10C are views of an extractor module 4200 according to the present disclosure. The extractor module 4200 can be utilized with any drawer described herein. The extractor module 4200 can include a scissor lift design. The scissor lift can include a platform or plate 4202. The plate 4202 can be coupled to a plurality of magnets 4204. The magnets 4204 can apply a magnetic field to the extraction containers 4026 of the extraction container holder 4020 as described herein. The plate 4202 can include magnets 4204 configured to apply a magnetic field to extraction containers 4026 positioned in a single lane. The plate 4202 can include magnets configured to apply a magnetic field to a single extraction container holder 4020. The scissor lift can include a base 4206. The base 4206 and the plate 4202 can be structures having the same or similar shape. The base 4206 and the plate 4202 can have a mirror image configuration. The scissor lift can move in the vertical direction. The scissor lift can include scissor legs 4208. The scissor legs 4208 are the crisscross struts that bridge the gap between the base 4206 and the plate 4202. One of the scissor legs 4208 is fixed to the plate 4202 and movable relative to the base 4206. The other scissor leg 4208 is fixed to the base 4206 and movable relative to the plate 4202. The scissor lift moves upward by extending the scissor legs 4208 from a generally horizontal orientation toward a generally vertical orientation. The scissor lift moves downward by extending the scissor legs 4208 from a generally vertical orientation toward a generally horizontal orientation. The extractor module 4200 can include the slide 4270. The slide 4270 can allow the extractor module 4200 to move relative to the corresponding drawer. The extractor module 4200 can include a linear drive 4272. The linear drive 4272 can move the extractor module 4200 relative to the slide 4270.

FIG. 10B illustrates the extractor module 4200 relative to the two lane drawer 4130. FIG. 10B shows three of the two lane drawers 4130. The extractor module 4200 can be used with any of the drawers described herein. The extractor module 4200 can be used with a three lane drawer 4170. The extractor module 4200 can be used with a six lane drawer 4190. The two lane drawers 4130 can include the extraction container station 4136. The extraction container station 4136 can hold two extraction container holders 4020. The extraction container holders 4020 can be side-by-side. The extractor module 4200 can be in a first position to apply a magnetic field to a first extraction container holder 4020 of the first two lane drawer 4130, as shown in FIG. 10B. The extractor module 4200 can move up and down relative to the first extraction container holder 4020 in the first position. The extractor module 4200 can apply a magnetic field to a single extraction container holder 4020 in the extraction container station 4136 of the first two lane drawer 4130. The extractor module 4200 can move from the first position to a second position. The extractor module 4200 can be in the second position to apply a magnetic field to a second extraction container holder 4020 of the first two lane drawer 4130. The extractor module 4200 can move along the slide 4270 to the second extraction container holder 4020. The extractor module 4200 can move up and down relative to the second extraction container holder 4020 in the second position.

The extractor module 4200 can move along the slide 4270 to the second two lane drawer 4130. The extractor module 4200 can move from the second position to a third position. The extractor module 4200 can be in the third position to apply a magnetic field to a third extraction container holder 4020 of the second two lane drawer 4130. The extractor module 4200 can move along the slide 4270 to the third extraction container holder 4020. The extractor module 4200 can move up and down relative to the third extraction container holder 4020 in the third position. The extractor module 4200 can move from the third position to a fourth position. The extractor module 4200 can be in the fourth position to apply a magnetic field to a fourth extraction container holder 4020 of the second two lane drawer 4130. The extractor module 4200 can move along the slide 4270 to the fourth extraction container holder 4020. The extractor module 4200 can move up and down relative to the fourth extraction container holder 4020 in the fourth position.

The extractor module 4200 can move along the slide 4270 to the third two lane drawer 4130. The extractor module 4200 can move from the fourth position to a fifth position. The extractor module 4200 can be in the fifth position to apply a magnetic field to a fifth extraction container holder 4020 of the third two lane drawer 4130. The extractor module 4200 can move along the slide 4270 to the fifth extraction container holder 4020. The extractor module 4200 can move up and down relative to the fifth extraction container holder 4020 in the fifth position. The extractor module 4200 can move from the fifth position to a sixth position. The extractor module 4200 can be in the sixth position to apply a magnetic field to a sixth extraction container holder 4020 of the third two lane drawer 4130. The extractor module 4200 can move along the slide 4270 to the sixth extraction container holder 4020. The extractor module 4200 can move up and down relative to the sixth extraction container holder 4020 in the sixth position. The extractor module 4200 can move from the sixth position back to the first position, and between any two of the first, second, third, fourth, fifth, or sixth positions. While the extractor module 4200 is shown in relation to the three of the two lane drawers 4130, the extractor module 4200 can be utilized with any drawer described herein.

FIG. 10A shows the extractor module 4200 in an extended or up configuration. FIG. 10C shows the extractor module 4200 in a collapsed or down configuration. The extractor module 4200 can move up and down to change the position of the magnetic field relative to the extraction containers 4026 of the extraction container holder 4020. The assembly can include three of the two lane drawers 4130. The two lane drawers 4130 can be positioned side-by-side. In some embodiments, each two lane drawers 4130 can include the extractor module 4200. The assembly can include three extractor modules 4200. The extractor module 4200 can move between lanes of the two lane drawers 4130. In some embodiments, the assembly can include one extractor module 4200. The extractor module 4200 can move between all of the two lane drawers 4130. The extractor module 4200 can move between three of the two lane drawers 4130. The extractor module 4200 can move between all six lanes. In some methods of use, the extractor module 4200 can be collapsed relative to the first two lane drawer 4130 after applying magnetic field to the extraction container holders 4020 in a first two lane drawer 4130. Then the extractor module 4200 can move to a second two lane drawer 4130, then extend to apply a magnetic field to the extraction container holders 4020 in the second two lane drawer 4130, and then collapsed relative to the second two lane drawer 4130. Then the extractor module 4200 can move to a third two lane drawer 4130, then extend to apply a magnetic field to the extraction container holders 4020 in the third two lane drawer 4130, and then collapsed relative to the third two lane drawer 4130. The extractor module 4200 can move to any two lane drawer 4130 in any order.

FIG. 11 is a view of an extractor module 4300 according to the present disclosure. The extractor module 4300 can be utilized with any drawer described herein. The extractor module 4300 can include a pusher design. The pusher can include a platform or plate 4302. The plate 4302 can be coupled to a plurality of magnets 4304. The plate 4302 can include magnets 4304. The magnets 4304 apply a magnetic field to extraction containers 4020. The magnets 4304 can be arranged to apply a magnetic field to two lanes. The magnets 4304 can be arranged to apply a magnetic field two extraction container holders 4020. In some embodiments, the plate 4302 with the magnets 4304 can stay in the two lane drawer 4130 at all times. In some embodiments, there can be one plate 4302 with the magnets 4304 in each two lane drawer 4130. The extractor module 4300 can include a pusher arm 4308. The pusher arm 4308 can rotate counterclockwise as viewed in FIG. 11 to move the plate 4302 upward and rotate clockwise as viewed in FIG. 11 to move the plate 4302 downward. The drawer 4130 can includes a plate 4310 having a slot 4312 through which the pusher arm 4308 rotates. The extractor module 4300 can include a slide 4370. The slide 4370 can allow the extractor module 4300 to move relative to the corresponding drawer. The extractor module 4300 can include a linear drive 4372. The linear drive 4372 can move the extractor module 4300 relative to the slide 4370. In some embodiments, the magnets 4304 are not directly attached to the linear drive 4372 or the pusher arm 4308.

FIG. 11 illustrates the extractor module 4300 relative to the two lane drawer 4130. The two lane drawer 4130 includes the pipette tip station 4134, the extraction container station 4136, and the amplification plate station 4138. The pipette tip station 4134 is configured to hold two pipette tip holders 4060. The extraction container station 4136 is configured to hold two extraction container holders 4020. The amplification plate station 4138 is configured to hold two amplification plates 4040. The extraction containers 4020 can be side-by-side. The extractor module 4300 can be in an up position to apply a magnetic field to a first extraction container holder 4020 and a second extraction container holder 4020. The extractor module 4300 can move up and down relative to the extraction container holders 4020. The extractor module 4300 can apply a magnetic field to two extraction container holders 4020 in the extraction container station 4136.

Referring back to FIG. 6C, the assembly can include three of the two lane drawers 4130. The two lane drawers 4130 can be positioned side-by-side. The extractor module 4300 can be collapsed relative to the first two lane drawer 4130. The extractor module 4300 can move to a second two lane drawer 4130. The extractor module 4300 can move along the slide 4370 to the second two lane drawer 4130. The extractor module 4200 can be in a second position to apply a magnetic field to the extraction container holders 4020 of the second two lane drawer 4130. The extractor module 4300 can move up and down relative to the second two lane drawer 4130 in the second position. The extractor module 4300 can be collapsed relative to the second two lane drawer 4130. The extractor module 4300 can move to a third two lane drawer 4130. The extractor module 4300 can move along the slide 4370 to the third two lane drawer 4130. The extractor module 4200 can be in a third position to apply a magnetic field to the extraction container holders 4020 of the third two lane drawer 4130. The extractor module 4300 can move up and down relative to the third two lane drawer 4130 in the third position. While the extractor module 4300 is shown in relation to the two lane drawer 4130, the extractor module 4300 can be utilized with any drawer described herein. The extractor module 4300 can move between drawers. The extractor module 4300 can reduce the total number of extractor modules needed in a multi-drawer assembly of the analyzer 4000. The extractor module 4300 can reduce costs. The extractor module 4300 can reduce complexity in manufacturing and complexity. This can allow one extractor module 4300 to reside in the analyzer 4000.

FIGS. 12A-12F depict a plate sealer 4400 according to the present disclosure. The plate sealer 4400 can have any of the features of the plate sealer 4220 shown in FIG. 3. The plate sealer 4400 can be located at the rear left corner of the analyzer 4000, similar to the plate sealer 4220. The plate sealer 4400 can have an opening that receives an inoculated amplification plate 4040.

Although the plate sealer 4400 can be located in the rear of analyzer 4000, the sealer 4400 can be accessed from the front of analyzer 4000 for replenishment of seal material. The ability to access components in the rear of analyzer 4000 through the front of analyzer 4000 allows analyzer 4000 to be placed directly against a wall in a laboratory. This placement of the analyzer 4000 can help to conserve floor space. To facilitate frontal access, the plate sealer can include one or more panels 4403 that can be removed. The panel 4403 is removed in FIG. 12B. The panel 4403 allows the plate sealer 4400 to be accessed from the front of the analyzer 4000.

The plate sealer 4400 is configured to bond a clear, optical seal to the top of the amplification plate 4040. The plate sealer 4400 is configured to seal multiple amplification plates during a processing cycle. In some embodiments, the plate sealer 4400 is configured to seal one amplification plate 4040 at a time. In some embodiments, the plate sealer 4400 is configured to seal a plurality of amplification plates 4040 per day. The plate sealer 4400 can be configured to seal a number of amplification plates 4040 per day, such as 6, 12, 18, 24, at least 6, at least 12, at least 18, at least 24, or any range of two of the foregoing values. In some embodiments, the plate sealer 4400 is configured to seal six amplification plates 4040 sequentially, corresponding to six plates loaded in the drawers in a non-limiting embodiment. In order to provide sealing capability for multiple amplification plates 4040 upon a single load, the sealer 4400 utilizes a roll-based seal material. The seal material can be provided by a single roll 4404. The seal material can be a film. The seal material can be a qPCR film. The seal material can be sheet of material. The seal material can be optically transparent. The seal material can be foil. The seal material can include a polypropylene layer. The seal material can be foil with a polypropylene layer. The plate sealer 4400 can apply seal material to components other than the amplification plate 4040. The plate scaler 4400 can apply seal material to any container. The single roll 4404 can be any length of material including 100 meters, 200 meters, 300 meters, 400 meters, 500 meters, 600 meters, 700 meters, 800 meters, 900 meters, 1000 meters, 1100 meters, 1200 meters, or any range of two of the foregoing values. The single roll 4404 can be loaded at one time. The single roll 4404 can be utilized for a plurality of processing cycles without needing to be replaced. The single roll 4404 can last numerous days, weeks, or months with regular processing. The single roll 4404 can be sufficient to seal amplification plates 4040 for a full year for most applications.

The plate sealer 4400 can include a sensor 4406. The sensor 4406 can be a low film sensor. The sensor 4406 can be configured to sense when the amount of seal material drops beneath a certain threshold level indicating that the single roll 4404 should be replaced. The sensor 4406 can be an optical sensor. The sensor 4406 can be mounted relative to the single roll 4404.

The plate sealer 4400 can include a rear housing 4408. The rear housing 4408 can be positioned near the rear of the analyzer 4000. The rear housing 4408 can include support for one or more internal components of the plate sealer 4400. The plate sealer 4400 can include a roll holder 4410. The roll holder 4410 can hold the single roll 4404 relative to the rear housing 4408. The roll holder 4410 can allow the single roll 4404 to rotate to feed seal material as described herein.

The plate sealer 4400 can include an idler roller 4412. The idler roller 4412 can be positioned relative to the rear housing 4408. The idler roller 4412 can be a simple conveyor roller. The idler roller 4412 can be configured to support the seal material as the seal material is moved in position. The idler roller 4412 can change the direction of the seal material from a generally vertical orientation to a generally horizontal orientation. The idler roller 4412 can be important for the smooth and efficient movement of the seal material. The idler roller 4412 can allow the single roll 4404 to feed seal material as described herein.

The plate sealer 4400 can include a feed plate 4414. The feed plate 4414 can advance the seal material. The feed plate 4414 can pull the seal material. The seal material can have sufficient rigidity to be pulled by the feed plate 4414 without stretching. The feed plate 4414 can pull the seal material causing the single roll 4404 to rotate relative to the roll holder 4410. The feed plate 4414 can pull the seal material causing the seal material and the idler roller 4412 to rotate.

The plate sealer 4400 can include a rear clamp assembly 4416. In some embodiments, the rear clamp assembly 4416 is movable. In some embodiments, the rear clamp assembly 4416 can move simultaneously with the feed plate 4414. In some embodiments, the rear clamp assembly 4416 can move independently of the feed plate 4414. The plate sealer 4400 can include a front clamp assembly 4418. In some embodiments, the front clamp assembly 4418 can be stationary.

The rear clamp assembly 4416 can clamp a portion of the seal material. The feed plate 4414 can advance the seal material until the seal material reaches the front clamp assembly 4418. The rear clamp assembly 4416 can advance with the feed plate 4414 while the seal material is clamped by the rear clamp assembly 4416. The front clamp assembly 4418 can clamp a portion of the seal material. The rear clamp assembly 4416 can open while the front clamp assembly 4418 clamps the seal material. The feed plate 4414 can retract while the front clamp assembly 4418 clamps the seal material. The rear clamp assembly 4416 can retract while the front clamp assembly 4418 clamps the seal material. The rear clamp assembly 4416 can clamp a portion of the seal material just behind the amplification plate 4040.

The rear clamp assembly 4416 and the front clamp assembly 4418 can clamp the seal material simultaneously. The rear clamp assembly 4416 and the front clamp assembly 4418 can clamp the seal material during sealing. The rear clamp assembly 4416 and the front clamp assembly 4418 can clamp the seal material during cutting. The rear clamp assembly 4416 and the front clamp assembly 4418 can clamp the seal material sequentially. The rear clamp assembly 4416 can clamp the seal material and then the front clamp assembly 4418 can clamp the seal material. The front clamp assembly 4418 can clamp the seal material and then the rear clamp assembly 4416 can clamp the seal material. The rear clamp assembly 4416 and the front clamp assembly 4418 can clamp the seal material independently. The rear clamp assembly 4416 and the front clamp assembly 4418 can clamp the seal material at different locations. The rear clamp assembly 4416 and the front clamp assembly 4418 can clamp the seal material at different times. The rear clamp assembly 4416 can clamp the seal material and move the seal material. The front clamp assembly 4418 can clamp the seal material at a single location. The rear clamp assembly 4416 can clamp the seal material at two locations. The seal material can be held between the rear clamp assembly 4416 and the front clamp assembly 4418.

The plate sealer 4400 can have a moveable platform 4402 that receives an inoculated amplification plate 4040. Wells in the inoculated amplification plate 4040 can contain fluids including extracted nucleic acids and reagents for amplification. The amplification plate 4040 can be deposited on the moveable platform 4402 of the plate sealer 4400. The amplification plate 4040 can be deposited by the multifunctional robot 4300 shown in FIG. 3. The multifunctional robot 4300 can be disengaged from the amplification plate 4040. The moveable platform 4402 can move the amplification plate 4040 into the plate sealer 4400. To apply the plate seal, the moveable platform 4402 positions the amplification plate 4040 within the plate sealer 4400. The moveable platform 4402 can position the amplification plate 4040 between the rear clamp assembly 4416 and the front clamp assembly 4418.

Turning now to FIG. 12C, the plate sealer 4400 can include a heated press plate 4420. The heated press plate 4420 can be positioned between the rear clamp assembly 4416 and the front clamp assembly 4418. The moveable platform 4402 can position the amplification plate 4040 under the heated press plate 4420. The heated press plate 4420 can be the same or similar shape as the amplification plate 4040. The heated press plate 4420 can be configured to apply heat to the entire top surface of the amplification plate 4040. The heated press plate 4420 can be configured to apply heat to the majority of the top surface of the amplification plate 4040. The heated press plate 4420 can be configured to apply pressure to the entire top surface of the amplification plate 4040. The heated press plate 4420 can be configured to apply pressure to the majority of the top surface of the amplification plate 4040.

The plate sealer 4400 can include a motor assembly 4422. The motor assembly 4422 can raise and lower the heated press plate 4420 relative to the amplification plate 4040. The motor assembly 4422 can facilitate the application of pressure to the heated press plate 4420.

The plate sealer 4400 can include a linear rail 4424. The feed plate 4414 can move along the linear rail 4424. The linear rail 4424 can guide the movement of the feed plate 4414 relative to the heated press plate 4420. The linear rail 4424 can guide the movement of the feed plate 4414 relative to the amplification plate 4040. The plate sealer 4400 can include additional rails to allow movement. An additional linear rail can guide the movement of the moveable platform 4402 into and out of the plate sealer 4400. The linear rail for the moveable platform 4402 can be mounted upside down. In some embodiments, the carriage or nest is fixed and the rail moves. The linear rail for the moveable platform 4402 can be located under the nest. The plate sealer 4400 can include a hinged door 4426. The hinged door 4426 can close when the moveable platform 4402 is disposed within the housing 4403, 4408. The hinged door 4426 can form an enclosure to prevent unwanted material from entering the plate sealer 4400.

The plate sealer 4400 can include a knife assembly 4430. The knife assembly 4430 can cut the seal material. The knife assembly 4430 can cut the seal material simultaneously with the seal material being bonded to the amplification plate 4040. The knife assembly 4430 can be lowered relative to the seal material. The knife assembly 4430 can be lowered relative to the amplification plate 4040. The knife assembly 4430 can lower with the heated press plate 4420. In some embodiments, there is a stationary knife 4431 in addition to the knife assembly 4430 that moves. The knife assembly 4430 and the stationary knife 4431 can shear the seal material. The action of cutting the seal material can be combined with sealing the seal material. The rear clamp assembly 4416 can be positioned behind the knife assembly 4430. The rear clamp assembly 4416 can clamp the seal material as the knife assembly 4430 cuts the material. The rear clamp assembly 4416 can clamp the free end of the seal material as the seal material is cut. The feed plate 4414 can maintain contact with the seal material as the knife assembly 4430 cuts the material. The cut end of the single roll 4404 is held in a position by the rear clamp assembly 4416 to allow repeated sealing operations.

The plate sealer 4400 can include a spring loaded nest assembly 4432. The spring loaded nest assembly 4432 moves the amplification plate 4040 which includes the plate body 4041. The body 4041 can be arranged on a nest 4043. The spring loaded nest assembly 4432 can bias the amplification plate 4040 and the nest 4043 upward toward the heated press plate 4420.

Once the eluted DNA is transferred into the amplification plate 4040, the amplification plate 4040 is moved to plate sealer 4400 where it is sealed. The analyzer 4000 uses the automated plate sealer 4400 to seal the amplification plate 4040 following elution. To transport the amplification plate 4040, the multifunctional robot 4300 is positioned such that a gripper mechanism hovers over the amplification plate 4040. The gripper arms are opened, the gripper is lowered, and the gripper arms close to engage the amplification plate 4040. The sensors in the gripper arms indicate when the gripper has engaged the amplification plate 4040. Once engaged, the amplification plate 4040 is lifted and transported by the robot to the moveable platform 4402. The moveable platform 4402 can include the nest 4043 upon which the amplification plate 4040 is positioned. The moveable platform 4402 moves relative to the linear rail 4424 into the plate sealer 4404. The moveable platform 4402 positions the amplification plate 4040 relative to rear clamp assembly 4416 and the front clamp assembly 4418. The moveable platform 4402 positions the amplification plate 4040 relative to the heated press plate 4420. The moveable platform 4402 positions the amplification plate 4040 relative to the knife assembly 4430. The hinged door 4426 can close.

The feed plate 4414 is configured to feed a section of seal material from the single roll 4404. The feed plate 4414 moves the seal material relative to the heated press plate 4420. The feed plate 4414 moves the seal material relative to knife assembly 4430. The feed plate 4414 moves the seal material relative to the rear clamp assembly 4416 and the front clamp assembly 4418. The seal material can move prior to the amplification plate 4040 being moved in position. The seal material can move after the amplification plate 4040 is moved in position. The seal material can move simultaneously with the amplification plate 4040 being moved in position.

The amplification plate 4040 is moved into position for a sealing operation. The amplification plate 4040 is below the heated press plate 4420. The amplification plate 4040 is between the rear clamp assembly 4416 and the front clamp assembly 4418. In some embodiments, the rear clamp assembly 4416 closes on the seal material. The seal material is clamped in at least one location during movement. In some embodiments, the rear clamp assembly 4416 and the feed plate 4414 move together. In some embodiments, the rear clamp assembly 4416 and the feed plate 4414 move forward. The rear clamp assembly 4416 and the feed plate 4414 move forward to feed the seal material over the top of the amplification plate 4040. The rear clamp assembly 4416 and the feed plate 4414 move toward the front clamp assembly 4418. The front clamp assembly 4418 is open when the rear clamp assembly 4416 and the feed plate 4414 move. The front clamp assembly 4418 clamps front edge of seal material. The rear clamp assembly 4416 opens releasing seal material. The rear clamp assembly 4416 and the feed plate 4414 retract leaving the seal material in place. The rear clamp assembly 4416 closes to capture the seal material just behind the amplification plate 4040.

The rear clamp assembly 4416 and the front clamp assembly 4418 can be actuated to clamp the seal material. The seal material can be clamped by the rear clamp assembly 4416 prior to the amplification plate 4040 being moved in position. The seal material can be clamped by the front clamp assembly 4418 after the amplification plate 4040 is moved in position. The seal material can be clamped by the rear clamp assembly 4416 after the amplification plate 4040 is moved in position. The seal material can be clamped simultaneously by the rear clamp assembly 4416 and the front clamp assembly 4418 after the amplification plate 4040 is moved in position. The seal material can be clamped simultaneously by the rear clamp assembly 4416 and the front clamp assembly 4418 while the amplification plate 4040 is moved in position.

The heated press plate 4420 can be heated. The heated press plate 4420 can be heated before the amplification plate 4040 is moved in position. The heated press plate 4420 can be heated after the amplification plate 4040 is moved in position. The heated press plate 4420 can be heated while the amplification plate 4040 is moved in position. The heated press plate 4420 can be lowered relative to the seal material. The seal material is positioned over the amplification plate 4040 when the heated press plate 4420 is lowered. The seal material is clamped between the rear clamp assembly 4416 and the front clamp assembly 4418 when the heated press plate 4420 is lowered. The heated press plate 4420 can be lowered relative to the amplification plate 4040, thereby pressing the seal material onto the top surface of the amplification plate 4040. The heated press plate 4420 is configured to use heat and pressure to bond the seal material to amplification plate 4040.

The knife assembly 4430 lowers with the heated press plate 4420. The knife assembly 4430 cuts the seal material. The knife assembly 4430 cuts material that extends beyond the amplification plate 4040. The knife assembly 4430 cuts between the rear clamp assembly 4416 and the front clamp assembly 4418. The rear clamp assembly 4416 clamps the free end of the seal material as the seal material is being cut. The rear clamp assembly 4416 maintains the position of the free end of the seal material. The rear clamp assembly 4416 enables continuous feeding of the seal material. After sealing, the amplification plate 4040 is moved out of the plate sealer 4400. The hinged door 4426 can open. The moveable platform 4402 moves relative to the linear rail 4424 out of the plate sealer 4400. The sealed amplification plate 4040 is available for transport within the analyzer 4000.

The analyzer 4000 uses the automated plate sealer 4400 to seal the amplification plate 4040 prior to plate mixing. The analyzer 4000 uses the automated plate sealer 4400 to seal the amplification plate 4040 prior to target amplification. The analyzer 4000 uses the automated plate scaler 4400 to seal the amplification plate 4040 prior to detection.

The plate sealer 4400 can utilize a roll of seal material. The seal material can be a film. The plate sealer 4400 can be configured to heat seal a length of the seal material to the top of the amplification plate 4040. The amplification plate 4040 can be an HPV PCR plate. The seal material is sealed to the amplification plate 4040 using a combination of heat and pressure. Each of the 96 wells of the amplification plate 4040 can be individually sealed. The seal material can completely encircle the open end of the well. The seal material can prevent contamination between wells of the amplification plate 4040. The seal material can prevent evaporation during amplification. The seal material can be optically transparent to allow for detection.

The plate sealer 4400 can be a self-contained assembly. The plate sealer 4400 can be a sub-assembly that is powered with AC power and communicates to software through a serial interface. The software can provide high level commands to the processor inside the plate sealer 4400. The plate sealer 4400 can include the roll holder 4410 with the sensor 4406. The sensor 4406 can detect when the seal material is low. The plate sealer 4400 can include a sensor for detecting seal material over the amplification plate 4040. The sensor can be near the knife assembly 4430. The sensor can be pointed toward the amplification plate 4040. The plate sealer 4400 can include the idler roller 4412. The plate sealer 4400 can include guides to control the seal material. The plate sealer 4400 can include the moveable platform 4402. The moveable platform 4402 can function as a drawer to extend the nest 4043 in and out of the scaler so amplification plate 4040 can be picked up by the multifunctional robot 4300. The plate sealer 4400 can include the feed plate 4414 to feed the seal material over the amplification plate 4040. The plate sealer 4400 can include the rear clamp assembly 4416 for holding the seal material against the amplification plate 4040) when the seal material is advanced. The plate sealer 4400 can include the front clamp assembly 4418 for holding the seal material against the amplification plate 4040 while the feed plate 4414 retracts. The plate sealer 4400 can include the heated press plate 4420 for sealing the seal material against the amplification plate 4040. The plate sealer 4400 can include the knife assembly 4430 for cutting the seal material. The plate sealer 4400 can include an internal control system. The plate sealer 4400 can be automated to seal the amplification plate 4040. The plate sealer 4400 can reduce costs. The plate sealer can reduce complexity in manufacturing and servicing.

The plate sealer 4400 seals and cuts in the same operation. The knife assembly 4430 moves with the heated press plate 4420. The knife assembly 4430 and the heated press plate 4420 are lowered simultaneously. The knife assembly 4430 and the heated press plate 4420 cut the seal material and bond the seal material to the amplification plate 4040. The cutting of the seal material and sealing of the seal material occurs in one motion. The cutting of the seal material and sealing of the seal material occurs simultaneously. Sealing and cutting at the same time allows for a more streamlined workflow. Sealing and cutting at the same time allows for less complex hardware.

The plate sealer 4400 does not cut the seal material prior to sealing. The plate sealer 4400 does not handle a cut piece of seal material. It is noted that handling a cut piece of seal material is challenging. The plate sealer 4400 moves the free end of the seal material in position. The seal material is sealed to the amplification plate 4040 and a new free end is formed by the knife assembly 4430. The new free end of the seal material is held in position by the rear clamp assembly 4416 and/or the feed plate 4414. The seal material only has one free end. The seal material is continuously in position to seal another amplification plate 4040. In some embodiments, a longer seal material is utilized for each amplification plate 4040 to allow for the seal material to be cut with sealing.

There are many different ways to accomplish the motions required for the plate sealer 4400. The heated press plate 4420 can utilize an integrated ball screw actuator. The heated press plate 4420 can utilize a cam mechanism. The heated press plate 4420 can utilize a rack and pinion gear design. The heated press plate 4420 can utilize a timing belt design. The moveable platform 4402 can be actuated by integrated lead screw motor assemblies. In some embodiments, stepper motors can be used for the axes, but brushless DC motors could be used instead. The rear clamp assembly 4416 and the front clamp assembly 4418 can be actuated with solenoids, but this motion could be accomplished with lead screws. The amplification plate 4040 can moved in and out of the plate sealer 4400 to allow the multifunctional robot 4300 to pick it up. The plate sealer 4400 can utilize heat-sealing technology. The plate sealer 4400 can utilize roll handling technology.

In some embodiments, a workflow is provided. The plate sealer 4400 is powered on. The high-throughput system 00, such as the pre-analytical system 10, can include a processor and a memory storing instructions for operating the plate sealer 4400. The high-throughput system 00 instructs plate sealer 4400 to reach a set temperature for the heated plate 4420. The high-throughput system 00 instructs plate sealer 4400 to initiate plate sealing operations at a set temperature. The heated press plate 4420 can be heated to the set temperature. The heated press plate 4420 can take a few minutes to get to the set temperature. In some embodiments, the heated press plate 4420 is heated before the amplification plate 4040 is positioned within the plate scaler 4400.

In some embodiments, the hinged door 4426 can open. The moveable platform 4402 can extend outward. The moveable platform 4402 can include the nest 4043. The nest 4043 is configured to accept the amplification plate 4040. The multifunctional robot 4300 places the amplification plate 4040 on the nest 4043. The moveable platform 4402 can move inward toward the heated press plate 4420. In some embodiments, the heated press plate 4420 is at the set temperature before the amplification plate 4040 is moved inward on the moveable platform 4402. The hinged door 4426 can close. The moveable platform 4402 can position the amplification plate 4040 under the heated press plate 4420. The heated press plate 4420 can be at the set temperature.

In some embodiments, the feed plate 4414, the rear clamp assembly 4416, and the front clamp assembly 4418 undergo a series of steps to position the seal material. In some embodiments, the rear clamp assembly 4416 clamps the seal material. The feed plate 4414 moves forward to feed the seal material over the top of the amplification plate 4040. In some embodiments, the rear clamp assembly 4416 and the feed plate 4414 are coupled for movement. In some embodiments, the rear clamp assembly 4416 and the feed plate 4414 move independently. In some embodiments, the rear clamp assembly 4416 clamps the seal material before the feed plate 4414 moves forward. The feed plate 4414 moves forward to feed the seal material toward the front clamp assembly 4418.

Next, the front clamp assembly 4418 clamps front edge of seal material. In some embodiments, the rear clamp assembly 4416 clamps the seal material and the front clamp assembly 4418 clamp the seal material simultaneously. Next, the rear clamp assembly 4416 opens releasing seal material. The front clamp assembly 4418 remains clamped to the seal material. Next, the feed plate 4414 retracts leaving the seal material in place. In some embodiments, the rear clamp assembly 4416 retracts. The front clamp assembly 4418 remains clamped to the seal material. Next, the rear clamp assembly 4416 closes to capture the seal material just behind the amplification plate 4040. The rear clamp assembly 4416 closes just behind the knife assembly 4430. In some embodiments, the rear clamp assembly 4416 clamps the seal material and the front clamp assembly 4418 clamps the seal material simultaneously.

The heated press plate 4420 and the knife assembly 4430 lower onto amplification plate 4040 to cut and apply the seal material. The rear clamp assembly 4416 and the front clamp assembly 4418 clamp the seal material as the heated press plate 4420 is lowered. The heated press plate 4420 is configured to apply heat and pressure to the seal material to seal each well individually. The rear clamp assembly 4416 and the front clamp assembly 4418 clamp the seal material as the knife assembly 4430 is lowered. The knife assembly 4430 cuts the seal material to create a new free end. The knife assembly 4430 cuts the seal material as the heated press plate 4420 applies heat and pressure. The heated press plate 4420 raises back up. The knife assembly 4430 raises back up. In some embodiments, the rear clamp assembly 4416 clamps the seal material just behind the amplification plate 4040. The hinged door 4426 can open. The moveable platform 4402 can extend outward away from the heated press plate 4420. The sealed amplification plate 4040 is presented for the multifunctional robot 4300 for pickup.

In some embodiments, a plate sealer for use in a diagnostic testing apparatus is provided. The plate sealer can include a heated press plate configured to apply heat and pressure to a seal material. In some embodiments, the seal material is configured to seal against a top surface of an amplification plate. In some embodiments, the amplification plate comprises a plurality of wells that define amplification compartments for amplification. The plate sealer can include a knife assembly configured to cut the seal material. In some embodiments, the heated press plate and the knife assembly are configured to be lowered simultaneously relative to the amplification plate to cut the seal material and seal the seal material to the amplification plate.

In some embodiments, the plate sealer can include a roll of the seal material. In some embodiments, the plate sealer can include a rear clamp assembly and a front clamp assembly. In some embodiments, the rear clamp assembly and the front clamp assembly are configured to clamp the seal material when the heated press plate and the knife assembly are lowered. In some embodiments, the plate sealer can include a feed plate configured to advance the seal material. In some embodiments, the plate sealer can include a motor assembly configured to raise and lower the heated press plate and the knife assembly. In some embodiments, the plate sealer can include the amplification plate. In some embodiments, the plate sealer can include a multifunctional robot configured to position the amplification plate. In some embodiments, the plate sealer can include a movable platform configured to receive the amplification plate. In some embodiments, the plate sealer can include a hinged door.

In some embodiments, a two lane drawer assembly is provided. The two lane drawer assembly can include a two lane drawer. The two lane drawer can include a pipette tip station configured to house two pipette tip holders. The two lane drawer can include an extraction container station configured to house two extraction container holders. The two lane drawer can include an amplification plate station configured to house two amplification plates. The two lane drawer assembly can include an extractor module positioned relative to the extraction container station. In some embodiments, the extractor module comprises magnets configured to apply a magnetic field to extraction container holders of the two extraction container holders.

In some embodiments, the extractor module is configured to process samples from the one extraction container holder. In some embodiments, the extractor module is configured to move up and down relative to the two extraction container holders. In some embodiments, the extractor module is configured to move up and down along one or more rails. In some embodiments, the two lane drawer comprises a retention feature configured to extend over a portion of the pipette tip station and the extraction container station. In some embodiments, the two lane drawer comprises a latching mechanism comprising a push-to-close latch. In some embodiments, the two lane drawer comprises a kick-out mechanism configured to facilitate the sliding extension of the two lane drawer. In some embodiments, the two lane drawer comprises telescoping slides. In some embodiments, the two lane drawer assembly can include the two pipette tip holders, the two extraction container holders, and the two amplification plates. In some embodiments, the two lane drawer assembly can include three of the two lane drawers. In some embodiments, the two lane drawer assembly can include a tip drawer configured to house five pipette tip holders.

The foregoing description is intended to illustrate various aspects of the present technology. It is not intended that the examples presented herein limit the scope of the present technology. The technology now being fully described, it will be apparent to one of ordinary skill in the art that many changes and modifications can be made thereto without departing from the spirit or scope of the appended claims.

What is claimed is:

1. A plate sealer for use in a diagnostic testing apparatus, the plate sealer comprising:
a heated press plate configured to apply heat and pressure to a seal material, wherein the seal material is configured to seal against a top surface of an amplification plate, wherein the amplification plate comprises a plurality of wells that define amplification compartments for amplification; and
a knife assembly configured to cut the seal material,
wherein the heated press plate and the knife assembly are configured to be lowered simultaneously relative to the amplification plate to cut the seal material and seal the seal material to the amplification plate.

2. The plate sealer of claim 1, further comprising a roll of the seal material.

3. The plate sealer of claim 1, further comprising a rear clamp assembly and a front clamp assembly, wherein the rear clamp assembly and the front clamp assembly are configured to clamp the seal material when the heated press plate and the knife assembly are lowered.

4. The plate sealer of claim 1, further comprising a feed plate configured to advance the seal material.

5. The plate sealer of claim 1, further comprising a motor assembly configured to raise and lower the heated press plate and the knife assembly.

6. The plate sealer of claim 1, further comprising the amplification plate.

7. The plate sealer of claim 1, further comprising a multifunctional robot configured to position the amplification plate.

8. The plate sealer of claim 1, further comprising a movable platform configured to receive the amplification plate.

9. The plate sealer of claim 1, further comprising a hinged door.
